(19) United States
(12) Patent Application Publication
(10) Pub. No.: US 2025/0092443 A1
(43) Pub. Date: Mar. 20, 2025

(54) ROLLING CIRCLE AMPLIFICATION METHODS AND PROBES FOR IMPROVED SPATIAL ANALYSIS

(71) Applicant: 10x Genomics, Inc., Pleasanton, CA (US)

(72) Inventor: Hyemin OH, Pleasanton, CA (US)

(21) Appl. No.: 18/888,129

(22) Filed: Sep. 17, 2024

Related U.S. Application Data
(60) Provisional application No. 63/583,533, filed on Sep. 18, 2023.

(51) Int. Cl. C12Q 1/682; C12Q 1/25; C12Q 1/6804; C12Q 1/6844; C12Q 1/6876

(52) U.S. Cl. CPC C12Q 1/682; C12Q 1/25; C12Q 1/6804; C12Q 1/6844; C12Q 1/6876; G01N 2333/9015

(57) ABSTRACT

The present disclosure relates in some aspects to methods and compositions for analyzing a biological sample. In some aspects, provided herein are probes, methods, and kits for analyzing a processing a biological sample, wherein the processing comprises performing rolling circle amplification of a ribosomal RNA (rRNA)-targeting probe, and detecting a detection probe hybridized to a non-rRNA target analyte (e.g., nucleic acid) in the biological sample.

### CROSS REFERENCE TO RELATED APPLICATIONS

This application claims priority to U.S. Provisional Patent Application No. 63/583,533, filed Sep. 18, 2023, entitled “ROLLING CIRCLE AMPLIFICATION METHODS AND PROBES FOR IMPROVED SPATIAL ANALYSIS,” which is herein incorporated by reference in its entirety for all purposes.

### FIELD

The present disclosure relates in some aspects to methods and compositions for in situ detection and analysis of analytes in a biological sample, and more specifically for methods for improved spatial analysis.

### BACKGROUND

Detection of nucleic acids in situ is useful for many purposes, such as understanding the molecular basis of cell identity and developing treatment for diseases. However, achievement of high-accuracy nucleic acid detection (e.g., transcriptomic imaging) has been hindered by optical and physical crowding, limiting the sensitivity and resolution of detection. Improved methods are needed. Provided herein are methods and compositions that address such and other needs.

### SUMMARY

Methods to minimize or eliminate the effects of physical and optical crowding during detection and decoding of barcoded target analytes will improve in situ analysis techniques for detection of nucleic acid and non-nucleic acid analytes in biological samples. Provided herein in some aspects is a method for analyzing a biological sample, comprising: (a) contacting the biological sample with a detection probe or probe set, wherein the detection probe or probe set hybridizes to a target nucleic acid in the biological sample; (b) contacting the biological sample with a ribosomal RNA (rRNA)-targeting probe or probe set, wherein the rRNA-targeting probe or probe set hybridizes to a ribosomal RNA (rRNA) target sequence in the biological sample, and wherein the rRNA-targeting probe is a circular probe or the rRNA-targeting probe or probe set is a circularizable probe or probe set and the method comprises circularizing the rRNA-targeting probe or probe set; (c) performing rolling circle amplification of the rRNA-targeting probe or probe set to generate a non-detection rolling circle amplification product (RCP) in the biological sample; and (d) imaging the biological sample to detect an optical signal associated with the detection probe or probe set or a product thereof, without detecting an optical signal associated with the non-detection RCP. In some embodiments, the method does not comprise detecting the non-detection RCP using a fluorescently labeled probe. In some embodiments, the method does not comprise detecting the non-detection RCP.

Provided herein in some aspects is a method for analyzing a biological sample, comprising: (a) contacting the biological sample with a detection probe or probe set, wherein the detection probe or probe set binds to a target analyte in the biological sample; (b) contacting the biological sample with a ribosomal RNA (rRNA)-targeting probe or probe set, wherein the rRNA-targeting probe or probe set hybridizes to a ribosomal RNA (rRNA) target sequence in the biological sample, and wherein the rRNA-targeting probe is a circular probe or the rRNA-targeting probe or probe set is a circularizable probe or probe set and the method comprises circularizing the rRNA-targeting probe or probe set; (c) performing rolling circle amplification of the rRNA-targeting probe or probe set to generate a non-detection rolling circle amplification product (RCP) in the biological sample; and (d) imaging the biological sample to detect an optical signal associated with the detection probe or probe set or a product thereof, without detecting an optical signal associated with the non-detection RCP. In some embodiments, the target analyte is a target nucleic acid. In some embodiments, the detection probe or probe set hybridizes to the target nucleic acid. In some embodiments, the method does not comprise detecting the non-detection RCP using a fluorescently labeled probe. In any of the embodiments herein, the target analyte comprises a nucleic acid, a protein, or an oligonucleotide reporter. In any of the embodiments herein, the target analyte can be associated with a non-nucleic acid analyte. In any of the embodiments herein, the detection probe or probe set hybridizes to a nucleic acid. In any of the embodiments herein, the target analyte is RNA. In some embodiments, the target analyte is mRNA. In any of the embodiments herein, the detection probe or probe set hybridizes to RNA. In any of the embodiments herein, the detection probe or probe set binds to a protein. In any of the embodiments herein, the detection probe or probe set binds to an oligonucleotide reporter. In any of the embodiments herein, the detection probe or probe set binds to a non-nucleic acid analyte. In any of the embodiments herein, the target analyte can be a probe or probe set associated with a nucleic acid analyte or product thereof in the biological sample.

In any of the embodiments herein, the detection probe or probe set can be a circular probe. In any of the embodiments herein, the detection probe or probe set can be a circularizable probe or probe set, and the method can comprise circularizing the detection probe or probe set. In some embodiments, the target analyte is a target nucleic acid; the detection probe or probe set hybridizes to the target nucleic acid; and the detection probe or probe set is a circular probe, or the detection probe or probe set is a circularizable probe or probe set and the method comprises circularizing the detection probe or probe set. In any of the embodiments herein, the method can comprise performing rolling circle amplification of the circular or circularized detection probe or probe set to generate a detection RCP, and the optical signal detected in (d) can be an optical signal associated with the detection RCP.

In some aspects, provided herein is a method for analyzing a biological sample, comprising: (a) contacting the biological sample with a detection probe or probe set, wherein the detection probe or probe set hybridizes to a target nucleic acid in the biological sample, and wherein the detection probe is a circular probe or the detection probe or probe set is a circularizable probe or probe set and the method comprises circularizing the detection probe or probe set; (b) contacting the biological sample with a ribosomal RNA (rRNA)-targeting probe or probe set, wherein the rRNA-targeting probe or probe set hybridizes to a ribosomal RNA (rRNA) target sequence in the biological sample, and wherein the rRNA-targeting probe is a circular probe or the rRNA-targeting probe or probe set is a circularizable probe or probe set and the method comprises circularizing the rRNA-targeting probe or probe set; (c) performing rolling circle amplification of the detection probe or probe set to generate a detection rolling circle amplification product (RCP) in the biological sample, and performing rolling circle amplification of the rRNA-targeting probe or probe set to generate a non-detection RCP in the biological sample; and (d) imaging the biological sample to detect an optical signal associated with the detection RCP without detecting an optical signal associated with the non-detection RCP. In some aspects, provided herein is a method for analyzing a biological sample, comprising: (a) contacting the biological sample with a detection probe or probe set, wherein the detection probe or probe set hybridizes to a target analyte in the biological sample, wherein the target analyte is a target nucleic acid, and wherein the detection probe is a circular probe or the detection probe or probe set is a circularizable probe or probe set and the method comprises circularizing the detection probe or probe set; (b) contacting the biological sample with a ribosomal RNA (rRNA)-targeting probe or probe set, wherein the rRNA-targeting probe or probe set hybridizes to a ribosomal RNA (rRNA) target sequence in the biological sample, and wherein the rRNA-targeting probe is a circular probe or the rRNA-targeting probe or probe set is a circularizable probe or probe set and the method comprises circularizing the rRNA-targeting probe or probe set; (c) performing rolling circle amplification of the detection probe or probe set to generate a detection rolling circle amplification product (RCP) in the biological sample, and performing rolling circle amplification of the rRNA-targeting probe or probe set to generate a non-detection RCP in the biological sample; and (d) imaging the biological sample to detect an optical signal associated with the detection RCP without detecting an optical signal associated with the non-detection RCP. In some embodiments, the method does not comprise detecting the non-detection RCP using a fluorescently labeled probe. In some embodiments, the method does not comprise detecting the non-detection RCP.

In any of the embodiments herein comprising circularizing the detection probe or probe set, the detection probe or probe set can be circularized by ligation selected from the group consisting of enzymatic ligation, chemical ligation, template dependent ligation, and/or template independent ligation. In any of the embodiments herein, the ligation can be enzymatic ligation. The enzymatic ligation can comprise using a ligase having an RNA-templated DNA ligase activity and/or an RNA-templated RNA ligase activity. In any of the embodiments herein, the enzymatic ligation can comprise using a ligase selected from the group consisting of a Chlorella virus DNA ligase (PBCV DNA ligase), a T4 RNA ligase, a T4 DNA ligase, and a single-stranded DNA (ssDNA) ligase.

In any of the embodiments herein comprising generating a detection RCP, the detection RCP can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the imaging comprises detecting a signal associated with a fluorescently labeled probe that directly or indirectly binds to the detection RCP. In some embodiments, a sequence of the detection RCP is analyzed at a location in the biological sample or a matrix embedding the biological sample. In some embodiments, the sequence of the detection RCP is analyzed by sequential hybridization, sequencing by hybridization, sequencing by ligation, sequencing by synthesis, sequencing by binding, or a combination thereof. In some embodiments, the sequence of the detection RCP comprises one or more barcode sequences or complements thereof. In some embodiments, the one or more barcode sequences or complements thereof correspond to the target analyte. In some embodiments, the one or more barcode sequences or complements thereof correspond to the target nucleic acid.

In any of the embodiments herein, the rRNA may be at least 20% of total cellular RNA in the biological sample. In any of the embodiments herein, the biological sample can be a eukaryotic sample, and the rRNA may be selected from the group consisting of 5S rRNA, 18S rRNA, 28S rRNA, and 5.8S rRNA. In some embodiments, the rRNA is 18S rRNA. In some any of the embodiments herein, the biological sample can be a prokaryotic sample, and the rRNA can be 5S, 23S or 16S rRNA. In any of the embodiments herein, at least 1,000 molecules of the rRNA-targeting probe may hybridize to rRNAs per cell in the biological sample. In any of the embodiments herein, performing rolling circle amplification of the rRNA-targeting probe can comprise generating between about 5,000 to about 50,000 RCPs per cell, between about 7,500 to about 25,000 RCPs per cell, or between about 10,000 to 15,000 RCPs per cell from the molecules of the rRNA targeting probe. In any of the embodiments herein, performing rolling circle amplification of the rRNA-targeting probe can comprise generating between about 10,000 to 15,000 RCPs per cell from the molecules of the rRNA targeting probe.

In any of the embodiments herein, the method can comprise contacting the biological sample with a non-circularizable probe or probe set, wherein the non-circularizable probe or probe set comprises a hybridization region complementary to the rRNA target sequence or to a portion of the rRNA target sequence. In any of the embodiments herein, a 5′ end of the non-circularizable probe or probe set may lack a 5′ phosphate. In some embodiments, the non-circularizable probe or probe set has been treated with an alkaline phosphatase prior to contacting the biological sample.

In any of the embodiments herein, the rRNA-targeting probe or probe set can be contacted with the biological sample at a concentration of between about 0.05 and about 5 nM, between about 0.5 and about 2.5 nM, between about 0.5 and about 1 nM, or between about 0.1 and about 1 nM. In any of the embodiments herein, the rRNA-targeting probe or probe set can be contacted with the biological sample at a concentration of about 0.5 nM. In any of the embodiments herein, the non-circularizable probe or probe set can be contacted with the biological sample at a concentration of between about 0.05 and about 5 nM, between about 0.5 and about 2.5 nM, between about 0.5 and about 1 nM, or between about 0.1 and about 1 nM. In any of the embodiments herein, the non-circularizable probe or probe set can be contacted with the biological sample at a concentration of about 4.5 nM. In any of the embodiments herein, the rRNA-targeting probe or probe set and the non-circularizable probe or probe set can be contacted with the biological sample simultaneously. In any of the embodiments herein, the ratio of the rRNA-targeting probe or probe set to the non-circularizable probe or probe set can be between about 1:5 and about 1:20, or between about 1:8 and 1:12. In any of the embodiments herein, the ratio of the rRNA-targeting probe or probe set to the non-circularizable probe or probe set can be 1:4 or lower. In any of the embodiments herein, the ratio of the rRNA-targeting probe or probe set to the non-circularizable probe or probe set can be 1:6 or lower. In any of the embodiments herein, the ratio of the rRNA-targeting probe or probe set to the non-circularizable probe or probe set can be 1:10 or lower.

In any of the embodiments herein, the detection probe or probe set can comprise a barcode sequence or complement thereof that corresponds to the target analyte. In any of the embodiments herein, the detection probe or probe set can comprise a barcode sequence or complement thereof that corresponds to the target nucleic acid. In any of the embodiments herein, the method can comprise analyzing the barcode sequence or complement thereof in the detection probe or probe set or in the product of the detection probe or probe set at a location in the biological sample or a matrix embedding the biological sample. In any of the embodiments herein, the barcode sequence or complement thereof can be analyzed by sequential hybridization, sequencing by hybridization, sequencing by ligation, sequencing by synthesis, sequencing by binding, or a combination thereof.

In any of the embodiments herein, detecting the barcode sequence or complement thereof can comprise: contacting the biological sample with a universal pool of detectably labeled probes and a first pool of intermediate probes, wherein an intermediate probe of the first pool of intermediate probes comprises a hybridization region complementary to the barcode sequence or complement thereof and a reporter region complementary to a detectably labeled probe of the universal pool of detectably labeled probes; detecting a complex formed between the barcode sequence or complement thereof, the intermediate probes of the first pool of intermediate probes, and the detectably labeled probe; and removing the intermediate probe of the first pool of intermediate probes and the detectably labeled probe. In any of the embodiments herein, detecting the barcode sequence or complement thereof can further comprise: contacting the biological sample with the universal pool of detectably labeled probes and a second pool of intermediate probes, wherein an intermediate probe of the second pool of intermediate probes comprises a hybridization region complementary to the barcode sequence or complement thereof and a reporter region complementary to a detectably labeled probe of the universal pool of detectably labeled probes; and detecting a complex formed between the barcode sequence or complement thereof, the intermediate probe of the second pool of intermediate probes, and the detectably labeled probe. In any of the embodiments herein, the barcode sequence or complement thereof can be assigned a series of signal codes that identifies the barcode sequence or complement thereof, and detecting the barcode sequence or complement thereof can comprise decoding the barcode sequence or complement thereof by detecting the corresponding sequence of signal codes detected from sequential hybridization, detection, and removal of sequential intermediate probes and the universal pool of detectably labeled probes. In some embodiments, the series of signal codes are fluorophore signatures assigned to the corresponding barcode sequences or complements thereof. In any of the embodiments herein, the detectably labeled probes can be fluorescently labeled.

In any of the embodiments herein, the method can be for multiplex detection of different target analytes in the biological sample, wherein the method comprises contacting the biological sample with a panel of detection probes or probe sets that bind to different target analytes in the biological sample. In any of the embodiments herein, the method can be for multiplex detection of different target nucleic acids in the biological sample, wherein the method comprises contacting the biological sample with a panel of detection probes or probe sets that hybridize to different target nucleic acids in the biological sample.

In any of the embodiments herein, the rRNA-targeting probe or probe set can be circularized by ligation selected from the group consisting of enzymatic ligation, chemical ligation, template dependent ligation, and/or template independent ligation. In any of the embodiments herein, the rRNA-targeting probe or probe set can be circularized by enzymatic ligation. In some embodiments, the enzymatic ligation comprises using a ligase having an RNA-templated DNA ligase activity and/or an RNA-templated RNA ligase activity. In any of the embodiments herein, the enzymatic ligation can comprise using a ligase selected from the group consisting of a Chlorella virus DNA ligase (PBCV DNA ligase), a T4 RNA ligase, a T4 DNA ligase, and a single-stranded DNA (ssDNA) ligase.

In any of the embodiments herein, the non-detection RCP can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In any of the embodiments herein, the method can be performed without analyzing a sequence of the non-detection RCP at a location in the biological sample or a matrix embedding the biological sample. In any of the embodiments herein, the method can be performed without analyzing a sequence of the non-detection RCP by sequential hybridization, sequencing by hybridization, sequencing by ligation, sequencing by synthesis, sequencing by binding, or a combination thereof.

In any of the embodiments herein, the target analyte can be a cellular nucleic acid analyte or a product thereof. In any of the embodiments herein, the target nucleic acid can be a cellular nucleic acid analyte or a product thereof. In any of the embodiments herein, the target analyte can be associated with a non-nucleic acid analyte. In any of the embodiments herein, the target nucleic acid can be associated with a non-nucleic acid analyte. In some embodiments, the target analyte is an oligonucleotide reporter in a labeling agent that binds to the non-nucleic acid analyte. In some embodiments, the target nucleic acid is an oligonucleotide reporter in a labeling agent that binds to the non-nucleic acid analyte. In any of the embodiments herein, the labeling agent can be an antibody conjugated to the oligonucleotide reporter. In some embodiments, the non-nucleic acid analyte is a protein. In any of the embodiments herein, the target analyte can be RNA. In some embodiments, the target analyte is mRNA. In any of the embodiments herein, the target nucleic acid can be RNA. In some embodiments, the target nucleic acid is mRNA. In any of the embodiments herein, the target analyte can be a probe or probe set associated with a nucleic acid analyte or product thereof in the biological sample. In any of the embodiments herein, the target nucleic acid can be a probe or probe set associated with a nucleic acid analyte or product thereof in the biological sample.

In any of the embodiments herein, the biological sample can be a fixed and/or permeabilized biological sample. In any of the embodiments herein, the biological sample can be a tissue sample. In any of the embodiments herein, the biological sample can be a formalin-fixed, paraffin-embedded (FFPE) tissue sample, a frozen tissue sample, or a fresh tissue sample. In any of the embodiments herein, the tissue sample can be a tissue slice between about 1 μm and about 50 μm in thickness. In any of the embodiments herein, the tissue slice can be between about 5 μm and about 35 μm in thickness. In any of the embodiments herein, the tissue slice can be between about 10 μm and about 20 μm in thickness. In any of the embodiments herein, the biological sample can be crosslinked. In any of the embodiments herein, the biological sample can be embedded in a hydrogel matrix. In any of the embodiments herein, the biological sample can be cleared. In some embodiments, the biological sample is not embedded in a hydrogel matrix.

In some of any of the embodiments herein, the biological sample comprises cells, and the method results in an increase in the average cell area of between about 5% and 15%. In some embodiments, the increased cell area is in comparison to a control biological sample that is not contacted with the circular or circularizable rRNA-targeting probe or probe set. In some of any of the embodiments herein, the biological sample comprises cells, and the method results in an increase in sensitivity for detection of the target analyte of between about 10% to about 60% in the detection of the target analyte in the biological sample by the detection probe or probe set. In some of any of the embodiments herein, the biological sample comprises cells, and the method results in an increase in sensitivity for detection of the target nucleic acid of between about 10% to about 60% in the detection of the target nucleic acid in the biological sample by the detection probe or probe set. In some embodiments, the increased sensitivity is in comparison to a control biological sample that is not contacted with the circular or circularizable rRNA-targeting probe or probe set. In some embodiments, the ratio of circularizable rRNA-targeting probes to total rRNA-targeting probes (circularizable rRNA-targeting probes and competing non-circularizable rRNA targeting probes) is about 1:1, and the method results in an increase in sensitivity for detection of the target nucleic acid by 53%. In some embodiments, the ratio of circularizable rRNA-targeting probes to total rRNA-targeting probes (circularizable rRNA-targeting probes and competing non-circularizable rRNA targeting probes) is about 1:2, and the method results in an increase in sensitivity for detection of the target nucleic acid by 31%. In some embodiments, the ratio of circularizable rRNA-targeting probes to total rRNA-targeting probes (circularizable rRNA-targeting probes and competing non-circularizable rRNA targeting probes) is about 1:10, and the method results in an increase in sensitivity for detection of the target nucleic acid by 13%. In some embodiments, the increase in sensitivity is measured by an increase in detection of barcode sequences with a Q-score of greater or equal to 20, and/or the density of detected detection RCPs.

In some embodiments, the increased sensitivity is in comparison to a control biological sample that is not contacted with the circular or circularizable rRNA-targeting probe or probe set. In some embodiments, the increase in sensitivity is measured by an increase in detection of barcode sequences with a Q-score of greater or equal to 20, and/or the density of detected detection RCPs.

In any of the embodiments herein, the optical signal associated with the detection probe or probe set or a product thereof can be enhanced by signal amplification in the biological sample. In some embodiments, the signal amplification comprises hybridization chain reaction (HCR) on the detection probe or probe set or product thereof; linear oligonucleotide hybridization chain reaction (LO-HCR) on the detection probe or probe set or product thereof; branched oligonucleotide hybridization chain reaction on the detection probe or probe set or product thereof; primer exchange reaction (PER) on the detection probe or probe set or product thereof.

In some aspects, provided herein is a method for analyzing a biological sample comprising a cell, comprising: (a) contacting the biological sample with a detection probe or probe set, wherein the detection probe or probe set hybridizes to a target nucleic acid in the biological sample; (b) contacting the biological sample with a ribosomal RNA (rRNA)-targeting probe or probe set, wherein the rRNA-targeting probe or probe set hybridizes to a ribosomal RNA (rRNA) target sequence in the biological sample, and wherein the rRNA-targeting probe is a circular probe or the rRNA-targeting probe or probe set is a circularizable probe or probe set and the method comprises circularizing the rRNA-targeting probe or probe set; (c) performing rolling circle amplification of the rRNA-targeting probe or probe set to generate a non-detection rolling circle amplification product (RCP) in the biological sample; and (d) detecting the detection probe or probe set or a product thereof, wherein the volume of the cell after the rolling circle amplification of the rRNA-targeting probe or probe set (V2) is greater than the volume of the cell before the rolling circle amplification of the rRNA-targeting probe or probe set (V1). In some embodiments, V2 is at least 105%, at least 110%, at least 115%, at least 120%, at least 130%, at least 140%, at least 160%, or at least 200% of V1. In some embodiments, V2 is no more than 200%, no more than 190%, no more than 180%, no more than 170%, no more than 160%, or no more than 150% of V1.

In some aspects, provided herein is a method for analyzing a cell in a biological sample, comprising: (a) contacting the cell with a detection probe or probe set, wherein the detection probe or probe set binds to a target analyte in the cell; (b) contacting the cell with a ribosomal RNA (rRNA)-targeting probe or probe set, wherein the rRNA-targeting probe or probe set hybridizes to a ribosomal RNA (rRNA) target sequence in the cell, and wherein the rRNA-targeting probe is circular or the rRNA-targeting probe or probe set is circularizable and the method comprises circularizing the rRNA-targeting probe or probe set; (c) performing rolling circle amplification of the rRNA-targeting probe or probe set to generate a non-detection rolling circle amplification product (RCP) in the cell; and (d) detecting the detection probe or probe set or a product thereof in the cell. In some embodiments, the volume of the cell after the rolling circle amplification of the rRNA-targeting probe or probe set (V2) is greater than the volume of the cell before the rolling circle amplification of the rRNA-targeting probe or probe set (V1). In some embodiments, V2 is at least 105%, at least 110%, at least 115%, at least 120%, at least 130%, at least 140%, at least 160%, or at least 200% of V1. In some embodiments, V2 is no more than 200%, no more than 190%, no more than 180%, no more than 170%, no more than 160%, or no more than 150% of V1.

In some aspects, provided herein is a method for analyzing a biological sample comprising cells, comprising: (a) contacting the biological sample with a detection probe or probe set, wherein the detection probe or probe set binds to a target analyte in the biological sample; (b) contacting the biological sample with a ribosomal RNA (rRNA)-targeting probe or probe set, wherein the rRNA-targeting probe or probe set hybridizes to a ribosomal RNA (rRNA) target sequence in the biological sample, and wherein the rRNA-targeting probe is a circular probe or the rRNA-targeting probe or probe set is a circularizable probe or probe set and the method comprises circularizing the rRNA-targeting probe or probe set; (c) performing rolling circle amplification of the rRNA-targeting probe or probe set to generate a non-detection rolling circle amplification product (RCP) in the biological sample; and (d) detecting the detection probe or probe set or a product thereof. In some embodiments, the average area of the cells in the biological sample after the rolling circle amplification of the rRNA-targeting probe or probe set is greater than the average area of the cells in the biological sample before the rolling circle amplification of the rRNA targeting probe or probe set, or the average area of cells in a control biological sample. In some aspects, provided herein is a method for analyzing a biological sample comprising cells, comprising: (a) contacting the biological sample with a detection probe or probe set, wherein the detection probe or probe set hybridizes to a target nucleic acid in the biological sample; (b) contacting the biological sample with a ribosomal RNA (rRNA)-targeting probe or probe set, wherein the rRNA-targeting probe or probe set hybridizes to a ribosomal RNA (rRNA) target sequence in the biological sample, and wherein the rRNA-targeting probe is a circular probe or the rRNA-targeting probe or probe set is a circularizable probe or probe set and the method comprises circularizing the rRNA-targeting probe or probe set; (c) performing rolling circle amplification of the rRNA-targeting probe or probe set to generate a non-detection rolling circle amplification product (RCP) in the biological sample; and (d) detecting the detection probe or probe set or a product thereof, wherein the average area of the cells in the biological sample after the rolling circle amplification of the rRNA-targeting probe or probe set is greater than the average area of the cells in the biological sample before the rolling circle amplification of the rRNA targeting probe or probe set, or the average area of cells in a control biological sample.

In any of the preceding embodiments, the biological sample can be a tissue slice. In any of the preceding embodiments, the thickness of the tissue slice after the rolling circle amplification of the rRNA-targeting probe or probe set (T2) may be greater than the thickness of the tissue slice before the rolling circle amplification of the rRNA-targeting probe or probe set (T1). In some embodiments, T2 is at least 105%, at least 110%, at least 115%, at least 120%, at least 130%, at least 140%, or at least 200% of T1. In some embodiments, T2 is no more than 200%, no more than 190%, no more than 180%, no more than 170%, no more than 160%, or no more than 150% of T1.

In any of the preceding embodiments, the tissue slice can be a brain tissue slice. In some embodiments, the brain tissue slice comprises tissue from a region of a cerebellum and/or a region of a cerebral cortex. In some embodiments, the region from the cerebral cortex comprises a frontal lobe, temporal lobe, parietal lobe, and/or occipital lobe. In some embodiments, the brain tissue slice comprises tissue from an orbitofrontal area, a medial frontal area, a prefrontal cortex region, a premotor cortex, and/or a primary motor cortex. In some embodiments, the brain tissue slice comprises tissue from a primary somatosensory cortex and/or a primary visual cortex. In some embodiments, the brain tissue slice comprises tissue from a parahippocampal gyrus, uncus, hippocampus, temporal horn, and/or choroidal fissure. In some embodiments, the brain tissue slice comprises a dentate gyrus region, a CA3 region, and/or a CA1 region. In some embodiments, the area of the dentate gyrus region after the rolling circle amplification of the rRNA-targeting probe or probe set (A2) is greater than the area of the dentate gyrus region before the rolling circle amplification of the rRNA-targeting probe or probe set (A1). In some embodiments, the area of the tissue slice after the RCA of the rRNA-targeting probe or probe set (A2) is greater than the area of the tissue slice before RCA of the rRNA-targeting probe or probe set (A1). In some embodiments, A2 is at least 105%, at least 110%, at least 115%, at least 120%, at least 130%, at least 140%, at least 160%, or at least 200% of A1. In some embodiments, A2 is no more than 200%, no more than 190%, no more than 180%, no more than 170%, no more than 160%, or no more than 150% of A1.

In any of the preceding embodiments, T1 of the tissue slice can be between about 1 μm and about 50 μm. In any of the preceding embodiments, T1 can be between about 5 μm and about 35 μm. In any of the preceding embodiments, T1 can be between about 10 μm and about 20 μm.

In any of the preceding embodiments, the average area of the cells in the biological sample after the rolling circle amplification of the rRNA-targeting probe or probe set may be between 5% and 15% greater than the average area of the cells in the biological sample before the rolling circle amplification of the rRNA targeting probe or probe set, or the average area of cells in a control biological sample. In any of the preceding embodiments, the method may result in an increase in sensitivity for detection of the target analyte of between about 10% to about 60% in the detection of the target analyte in the biological sample by the detection probe or probe set. In any of the preceding embodiments, the method may result in an increase in sensitivity for detection of the target nucleic acid of between about 10% to about 60% in the detection of the target nucleic acid in the biological sample by the detection probe or probe set. In some embodiments, the increased sensitivity is in comparison to a control biological sample that is not contacted with the circular or circularizable rRNA-targeting probe or probe set. In some embodiments, the increase in sensitivity is measured by an increase in detection of barcode sequences with a Q-score of greater or equal to 20, and/or the density of detected detection RCPs generated from the detection probe or probe set.

In some embodiments, the target analyte is a target nucleic acid. In some embodiments, the detection probe or probe set hybridizes to the target nucleic acid. In any of the preceding embodiments, the detection probe or probe set can be a circular probe, or the detection probe or probe set can be a circularizable probe or probe set and the method can comprise circularizing the detection probe or probe set. In any of the preceding embodiments, the method can comprise performing rolling circle amplification of the circular or circularized detection probe or probe set to generate a detection RCP, and the detecting in (d) can be detecting the detection RCP. In any of the preceding embodiments, the detection probe or probe set can be circularized by ligation selected from the group consisting of enzymatic ligation, chemical ligation, template dependent ligation, and/or template independent ligation. In any of the preceding embodiments, the ligation can be enzymatic ligation, wherein the enzymatic ligation comprises using a ligase having an RNA-templated DNA ligase activity and/or an RNA-templated RNA ligase activity. In any of the preceding embodiments, the enzymatic ligation can comprise using a ligase selected from the group consisting of a Chlorella virus DNA ligase (PBCV DNA ligase), a T4 RNA ligase, a T4 DNA ligase, and a single-stranded DNA (ssDNA) ligase.

In any of the preceding embodiments, the detection RCP can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In any of the preceding embodiments, detecting the detection probe or probe set or product thereof can comprise detecting a signal associated with a fluorescently labeled probe that directly or indirectly binds to the detection RCP. In any of the preceding embodiments, the method can comprise analyzing a sequence of the detection RCP at a location in the biological sample or a matrix embedding the biological sample. In any of the preceding embodiments, the sequence of the detection RCP can be analyzed by sequential hybridization, sequencing by hybridization, sequencing by ligation, sequencing by synthesis, sequencing by binding, or a combination thereof. In any of the preceding embodiments, the sequence of the detection RCP can comprise one or more barcode sequences or complements thereof. In any of the preceding embodiments, the one or more barcode sequences or complements thereof may correspond to the target analyte. In any of the preceding embodiments, the one or more barcode sequences or complements thereof may correspond to the target nucleic acid.

In any of the preceding embodiments, the rRNA may be at least 20% of total cellular RNA in the biological sample. In any of the preceding embodiments, the biological sample can be a eukaryotic sample, and the rRNA can be selected from the group consisting of 5S rRNA, 18S rRNA, 28S rRNA, and 5.8S rRNA. In some embodiments, the rRNA is 18S rRNA. In any of the preceding embodiments, the biological sample can be a prokaryotic sample, and the rRNA can be 5S, 23S, or 16S rRNA. In any of the preceding embodiments, at least 1,000 or at least 5,000 molecules of the rRNA-targeting probe can be hybridized to rRNAs per cell in the biological sample. In any of the preceding embodiments, performing rolling circle amplification of the rRNA-targeting probe can comprise generating between about 5,000 to about 50,000 RCPs per cell, about 7,500 to about 25,000 RCPs per cell, or between about 10,000 to 15,000 RCPs per cell from the molecules of the rRNA targeting probe. In any of the preceding embodiments, performing rolling circle amplification of the rRNA-targeting probe can comprise generating between about 10,000 to 15,000 RCPs per cell from the molecules of the rRNA targeting probe.

In any of the preceding embodiments, the method can comprise contacting the biological sample with a non-circularizable probe or probe set, wherein the non-circularizable probe or probe set comprises a hybridization region complementary to the rRNA target sequence or to a portion of the rRNA target sequence. In some embodiments, a 5′ end of the non-circularizable probe or probe set lacks a 5′ phosphate. In some embodiments, the non-circularizable probe or probe set has been treated with an alkaline phosphatase prior to contacting the biological sample. In any of the preceding embodiments, method can be performed without detecting the non-detection RCP using a fluorescently labeled probe.

In any of the embodiments herein, the rRNA-targeting probe or probe set can be contacted with the biological sample at a concentration of between about 0.05 and about 5 nM, between about 0.5 and about 2.5 nM, between about 0.5 and about 1 nM, or between about 0.1 and about 1 nM. In any of the embodiments herein, the rRNA-targeting probe or probe set can be contacted with the biological sample at a concentration of about 0.5 nM. In any of the embodiments herein, the non-circularizable probe or probe set can be contacted with the biological sample at a concentration of between about 0.05 and about 5 nM, between about 0.5 and about 2.5 nM, between about 0.5 and about 1 nM, or between about 0.1 and about 1 nM. In any of the embodiments herein, the non-circularizable probe or probe set can be contacted with the biological sample at a concentration of about 4.5 nM. In any of the embodiments herein, the rRNA-targeting probe or probe set and the non-circularizable probe or probe set can be contacted with the biological sample simultaneously. In any of the embodiments herein, the ratio of the rRNA-targeting probe or probe set to the non-circularizable probe or probe set can be between about 1:5 and about 1:20, or between about 1:8 and 1:12. In any of the embodiments herein, the ratio of the rRNA-targeting probe or probe set to the non-circularizable probe or probe set is 1:4 or lower. In any of the embodiments herein, the ratio of the rRNA-targeting probe or probe set to the non-circularizable probe or probe set is 1:6 or lower. In any of the embodiments herein, the ratio of the rRNA-targeting probe or probe set to the non-circularizable probe or probe set is 1:10 or lower.

In any of the embodiments herein, the detection probe or probe set can comprise a barcode sequence or complement thereof that corresponds to the target analyte. In any of the embodiments herein, the detection probe or probe set can comprise a barcode sequence or complement thereof that corresponds to the target nucleic acid. In any of the embodiments herein, the method can comprise analyzing the barcode sequence or complement thereof in the detection probe or probe set or in the product of the detection probe or probe set at a location in the biological sample or a matrix embedding the biological sample. In any of the embodiments herein, the barcode sequence or complement thereof can be analyzed by sequential hybridization, sequencing by hybridization, sequencing by ligation, sequencing by synthesis, sequencing by binding, or a combination thereof. In any of the embodiments herein, detecting the barcode sequence or complement thereof can comprises: contacting the biological sample with a universal pool of detectably labeled probes and a first pool of intermediate probes, wherein an intermediate probe of the first pool of intermediate probes comprises a hybridization region complementary to the barcode sequence or complement thereof and a reporter region complementary to a detectably labeled probe of the universal pool of detectably labeled probes; detecting a complex formed between the barcode sequence or complement thereof, the intermediate probes of the first pool of intermediate probes, and the detectably labeled probe; and removing the intermediate probe of the first pool of intermediate probes and the detectably labeled probe. In any of the embodiments herein, detecting the barcode sequence or complement thereof can further comprise: contacting the biological sample with the universal pool of detectably labeled probes and a second pool of intermediate probes, wherein an intermediate probe of the second pool of intermediate probes comprises a hybridization region complementary to the barcode sequence or complement thereof and a reporter region complementary to a detectably labeled probe of the universal pool of detectably labeled probes; and detecting a complex formed between the barcode sequence or complement thereof, the intermediate probe of the second pool of intermediate probes, and the detectably labeled probe.

In any of the embodiments herein, the barcode sequence or complement thereof can be assigned a series of signal codes that identifies the barcode sequence or complement thereof. In any of the embodiments herein, detecting the barcode sequence or complement thereof can comprise decoding the barcode sequence or complement thereof by detecting the corresponding series of signal codes detected from sequential hybridization, detection, and removal of sequential intermediate probes and the universal pool of detectably labeled probes. In any of the embodiments herein, the series of signal codes can be fluorophore signatures assigned to the corresponding barcode sequences or complements thereof. In any of the embodiments herein, the detectably labeled probes can be fluorescently labeled. In any of the embodiments herein, the method can be for multiplex detection of different target analytes in the biological sample, and the method can comprise contacting the biological sample with a panel of detection probes or probe sets that bind to different target analytes in the biological sample. In any of the embodiments herein, the method can be for multiplex detection of different target nucleic acids in the biological sample, and the method can comprise contacting the biological sample with a panel of detection probes or probe sets that hybridize to different target nucleic acids in the biological sample.

In any of the embodiments herein, the rRNA-targeting probe or probe set can be circularized by ligation selected from the group consisting of enzymatic ligation, chemical ligation, template dependent ligation, and/or template independent ligation. In any of the embodiments herein, the rRNA-targeting probe can be circularized by enzymatic ligation, wherein the enzymatic ligation comprises using a ligase having an RNA-templated DNA ligase activity and/or an RNA-templated RNA ligase activity. In any of the embodiments herein, the enzymatic ligation can comprise using a ligase selected from the group consisting of a Chlorella virus DNA ligase (PBCV DNA ligase), a T4 RNA ligase, a T4 DNA ligase, and a single-stranded DNA (ssDNA) ligase.

In any of the embodiments herein, the non-detection RCP can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, and T3 RNA polymerase, or a variant or derivative of any of the foregoing polymerases.

In any of the embodiments herein, the method can be performed without analyzing a sequence of the non-detection RCP at a location in the biological sample or a matrix embedding the biological sample. In any of the embodiments herein, the method can be performed without analyzing a sequence of the non-detection RCP by sequential hybridization, sequencing by hybridization, sequencing by ligation, sequencing by synthesis, sequencing by binding, or a combination thereof.

In any of the embodiments herein, the target analyte can be a cellular nucleic acid analyte or a product thereof. In any of the embodiments herein, the target analyte can be associated with a non-nucleic acid analyte. In some embodiments, the target analyte is an oligonucleotide reporter in a labeling agent that binds to the non-nucleic acid analyte. In any of the embodiments herein, the target nucleic acid can be a cellular nucleic acid analyte or a product thereof. In any of the embodiments herein, the target nucleic acid can be associated with a non-nucleic acid analyte. In some embodiments, the target nucleic acid is an oligonucleotide reporter in a labeling agent that binds to the non-nucleic acid analyte. In any of the embodiments herein, the labeling agent can be an antibody conjugated to the oligonucleotide reporter, optionally wherein the non-nucleic acid analyte is a protein. In any of the embodiments herein, the target analyte can be RNA. In some embodiments, the target analyte is mRNA. In any of the embodiments herein, the target analyte can be a probe or probe set associated with a nucleic acid analyte or product thereof in the biological sample. In any of the embodiments herein, the target nucleic acid can be RNA. In some embodiments, the target nucleic acid is mRNA. In any of the embodiments herein, the target nucleic acid can be a probe or probe set associated with a nucleic acid analyte or product thereof in the biological sample.

In any of the embodiments herein, the detection probe or probe set and the rRNA-targeting probe or probe set can be contacted with the biological sample simultaneously. In any of the embodiments herein, the detection probe or probe set and the rRNA-targeting probe or probe set can be contacted with the biological sample sequentially, in either order.

In any of the embodiments herein, detecting the detection probe or probe set or a product thereof can comprise performing hybridization chain reaction (HCR) on the detection probe or probe set or product thereof; linear oligonucleotide hybridization chain reaction (LO-HCR) on the detection probe or probe set or product thereof; branched oligonucleotide hybridization chain reaction on the detection probe or probe set or product thereof; primer exchange reaction (PER) on the detection probe or probe set or product thereof.

In any of the embodiments herein, the detection probe or probe set can be a detection probe set comprising a plurality of detection probes that hybridize to a plurality of target sequences in the target nucleic acid. In some embodiments, the target analyte is a target nucleic acid, and the detection probe or probe set can be a detection probe set comprising a plurality of detection probes that hybridize to a plurality of target sequences in the target nucleic acid.

In some aspects, provided herein is a method of detecting a target analyte in a cell, comprising: (a) using a circular or circularizable ribosomal RNA (rRNA)-targeting probe or probe set to generate a plurality of non-detection rolling circle amplification products (RCPs) in the cell, thereby increasing the volume of the cell; and (b) detecting an optical signal associated with the target analyte. In any of the embodiments herein, the method does not comprise detecting an optical signal associated with the non-detection RCP. In any of the embodiments herein, the method comprises detecting a plurality of optical signals associated with one or more target analytes at a plurality of locations in the cell. In any of the embodiments herein, increasing the volume of the cell reduces optical crowding of the plurality of optical signals.

In some aspects, provided herein is a kit for analyzing a biological sample, the kit comprising: (a) a detection probe or probe set, wherein the detection probe or probe set comprises a target recognition sequence complementary to a target nucleic acid in the biological sample; (b) a ribosomal RNA (rRNA)-targeting probe or probe set, wherein the rRNA-targeting probe or probe comprises a targeting sequence complementary to a ribosomal RNA (rRNA) target sequence in the biological sample, and wherein the rRNA-targeting probe is a circular probe or a circularizable probe or probe set; and (c) a non-circularizable probe or probe set, wherein the non-circularizable probe or probe set comprises a hybridization region complementary to the rRNA target sequence or to a portion of the rRNA target sequence. In any of the embodiments herein, the detection probe or probe set can be a detection probe set comprising a plurality of detection probes capable of hybridizing to a plurality of target sequences in the target nucleic acid. In any of the embodiments herein, the detection probe or probe set can be a circular probe, or the detection probe or probe set can be a circularizable probe or probe set. In any of the embodiments herein, a 5′ end of the non-circularizable probe or probe set can lack a 5′ phosphate. In any of the embodiments herein, the non-circularizable probe or probe set can comprise a probe that has been treated with an alkaline phosphatase prior to contacting the biological sample.

In some aspects, provided herein is a kit for analyzing a biological sample, the kit comprising: (a) a detection probe or probe set, wherein the detection probe or probe set is capable of binding to a target analyte in the biological sample, and wherein the detection probe or probe set comprises a detectable label; (b) a ribosomal RNA (rRNA)-targeting probe or probe set, wherein the rRNA-targeting probe or probe set comprises a targeting sequence complementary to a ribosomal RNA (rRNA) target sequence in the biological sample, and wherein the rRNA-targeting probe is a circular probe or a circularizable probe or probe set; and (c) a non-circularizable probe or probe set, wherein the non-circularizable probe or probe set comprises a hybridization region complementary to the rRNA target sequence or to a portion of the rRNA target sequence. In some embodiments, the target analyte is a nucleic acid analyte or a non-nucleic acid analyte. In some embodiments, the target analyte is a protein. In some embodiments, the detection probe or probe set comprises an antibody.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings illustrate certain features and advantages of this disclosure. These embodiments are not intended to limit the scope of the appended claims in any manner.

FIG. 1 shows an example workflow of a method of analyzing a biological sample using generation of RCPs from rRNA-targeting probes to provide an expansion microscopy-like effect.

FIG. 2 shows quantification of average dentate gyrus (DG) area at various ratios of circularizable to total (circularizable and non-circularizable) 18S rRNA probe (left panel) and depicts fluorescent images of the negative control dentate gyrus (100% non-circularizable 18S rRNA-targeting probe, and treated with a phosphatase to ensure there were no phosphate group remaining in 5′ end) and positive control (100% circularizable 18S rRNA-targeting probe), showing the expanded morphology of cells that underwent RCA with circularizable 18S rRNA probes (right panel).

FIG. 3 characterizes the expanded morphology of cells that underwent RCA with 18S rRNA probes by quantifying the thickness of the dentate gyrus (DG), the CA3 region, and the curvature of the dentate gyrus (e.g., DG curving).

FIG. 4 depicts fluorescent images of tissue sections (e.g., tissue slices) that underwent RCA with various ratios of circularizable:total (e.g., circularizable and non-circularizable) 18S rRNA-targeting probes, costained with DAPI and HH3.

FIG. 5 shows the total number of objects (top) and fraction of objects that were detected in tissue sections (e.g., tissue slices) that underwent RCA with the mixture of 18S rRNA-targeting probes indicated and the panel of mRNA-targeting detection probes.

FIG. 6 depicts expression heatmaps showing distribution and density of decoded barcodes corresponding to Prox1 and Satb2 RNA, two representative mRNA targets from the detection probe panel with well-characterized expression patterns. Non-circularizable 18S rRNA-targeting probes only (negative control) and a mixture of circularizable and non-circularizable (0.5 nM circularizable: 4.5 nM non-circularizable) 18S rRNA-targeting probes were used.

FIG. 7 is an example analysis workflow of a biological sample (e.g., a cell or tissue sample) using an opto-fluidic instrument, according to various embodiments.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods and kits for improved spatial analysis of target analytes, such as nucleic acids, in a biological sample (e.g., tissue). The methods and kits provided herein can be applied to various applications such as in situ methods. Methods of analyzing the locations and identities of analytes in a biological sample can provide important information regarding cell types and functions in numerous contexts. However, performing such methods (e.g. for imaging the transcriptome in situ) with high accuracy has been hindered by diffraction limits of optical resolution to a few hundred nanometers, which is far greater than the size of many biological molecules (e.g., resulting in optical crowding); and which in many contexts provides insufficient resolution due to the high density of analytes (e.g. transcripts) found within biological samples, such as cells (e.g., physical crowding). In some aspects, optical crowding occurs when optical signals in a biological sample are generated at a density at which the optical signals overlap and/or cannot be easily distinguished from one another. Optical crowding can be especially problematic when a large number of target analytes are analyzed in a spatial context in a biological sample, necessitating the generation of a high density of corresponding optical signals in the sample. Furthermore, optical crowding of signals may result in decoding errors in certain in situ analysis assays, manifesting as large sensitivity and specificity losses, while physical crowding of rolling circle products (RCPs) may result in premature cessation of RCA amplification, limiting the number of transcripts per cell and hindering resolution of detection. Other imaging techniques have emerged (e.g., near-field imaging, far-field super-resolution microscopy) that may offer certain advantages for imaging of single molecules at nanoscale. However, these techniques have notable limitations, such as requiring expensive equipment and 3D super-resolution imaging and being too low-throughput (i.e., slow imaging speed) to be used to analyze a large number of samples and/or analytes, e.g. on a transcriptome-scale. In some embodiments, the methods provided herein can overcome these limitations by physically creating an expansion-microscopy-like effect within cells through RCA of a circular or circularizable probe or probe set that hybridizes to a highly abundant RNA (e.g., an rRNA) in the biological sample. In some aspects, the “expansion-microscopy-like effect” can be utilized to mitigate optical crowding of other probes targeting different analytes such as gene transcripts (and/or optical crowding of signals generated therefrom), thereby increasing sensitivity of in-situ sequencing. In some embodiments, RCPs may act as a crowding agent to improve resolution of spatial analysis in situ.

In some cases, the method also comprises contacting the biological sample with a non-circularizable probe or probe set that hybridizes to the target rRNA. In some embodiments, the non-circularizable probe or probe set targeting the rRNA competes with the circular or circularizable rRNA-targeting probe for hybridization to the target rRNA. The present disclosure provides data demonstrating that the combination of a non-circularizable probe or probe set comprising a hybridization region complementary to a target rRNA and a circular or circularizable rRNA-targeting probe or probe set comprising a hybridization region complementary to the target rRNA improves resolution and/or sensitivity for detecting a different target nucleic acid (e.g., mRNA) in the biological sample. In some aspects, providing the non-circularizable probe or probe set introduces control over the degree to which expansion occurs in the biological sample. For example, increasing the ratio of the non-circularizable rRNA-targeting probe to the circularizable rRNA-targeting probe reduces the degree to which the biological sample is expanded. In some aspects, the method comprises contacting the biological sample with a mixture of circularizable and non-circularizable probes targeting highly abundant rRNA targets, e.g., 18S rRNA. In some aspects, the method comprises contacting the biological sample with a mixture of circularizable and non-circularizable probes targeting highly abundant RNA targets.

Provided herein in some aspects are methods for RCP-based detection of analytes in a biological sample using a circularizable detection probe or probe set, wherein the detection probe or probe set hybridizes to a target nucleic acid in the biological sample, and a mixture of circularizable and non-circularizable ribosomal RNA (rRNA)-targeting probe or probe set, wherein the rRNA-targeting probe or probe set hybridizes to a ribosomal RNA (rRNA) target sequence in the biological sample; and imaging the biological sample to detect an optical signal associated with the detection probe or probe set or a product thereof, without detecting an optical signal associated with the non-detection RCP. In some embodiments, the ratio of the rRNA-targeting probe or probe set to the non-circularizable probe or probe set is between about 1:5 and about 1:20, or between about 1:8 and 1:12. In some embodiments, the ratio of the rRNA-targeting probe or probe set to the non-circularizable probe or probe set is 1:4 or lower, 1:6 or lower, or 1:10 or lower. In some embodiments, the rRNA-targeting probe or probe set is contacted with the biological sample at a concentration of between about 0.05 and about 5 nM, between about 0.5 nM and about 2.5 nM, between about 0.5 nM and about 1 nM, or between about 0.1 nM and about 1 nM. In some embodiments, the rRNA-targeting probe or probe set is contacted with the biological sample at a concentration of about 0.5 nM.

In some embodiments, the biological sample is contacted with a probe mixture with a ratio of circularizable rRNA targeting probe to total (circularizable and non-circularizable) rRNA targeting probes of 1:2, 1:4, 1:10, 1:25, 1:50, or any range having any of the forging ratios as endpoints in the range. In some embodiments, the ratio of circularizable rRNA-targeting probes to total rRNA-targeting probes (circularizable rRNA-targeting probes and competing non-circularizable rRNA targeting probes) is between 1:4 and 1:25, between 1:5 and 1:20, between 1:8 and 1:12, or about 1:10. In some embodiments, the ratio of circularizable rRNA-targeting probes to total rRNA-targeting probes (circularizable rRNA-targeting probes and competing non-circularizable rRNA targeting probes) is about 1:10.

In some embodiments, cellular morphology by RCA of the circularized rRNA-targeting probes is expanded. In some embodiments, RCA of the circularized rRNA-targeting probes facilitates cellular expansion without disrupting overall cellular morphology, e.g. such that the relative spatial relationships of cellular components to one another are maintained. In some embodiments, RCA of the circularized rRNA-targeting probes facilitates cellular expansion without disrupting overall tissue morphology, e.g. such that different components of the tissue remain in proportion to one another as in the tissue prior to the RCA of the circularized rRNA-targeting probes. In some embodiments, the cell nucleus remains intact. In some embodiments, performing RCA of circularized probes targeting rRNA achieves improved resolution of optical signals in situ, wherein the optical signals are associated with detection probes or probe sets targeting a target nucleic acid that is not the rRNA (e.g., the target nucleic acid is an mRNA). In some embodiments, performing RCA of circularized probes targeting rRNA achieves improved resolution of optical signals in situ, wherein the optical signals are associated with detection probes or probe sets targeting a target analyte that is not the target rRNA. The target analyte can be any suitable analyte, such as a nucleic acid analyte, or non-nucleic acid analyte such as a protein. In some embodiments, performing RCA of circularized probes targeting rRNA achieves improved resolution of optical signals in situ, wherein the optical signals are associated with RCPs generated from circular or circularized detection probes or probe sets targeting a target nucleic acid that is not the target rRNA (e.g., the target nucleic acid is an mRNA). In some embodiments, performing RCA of rRNA-targeting probes achieves improved resolution of optical signals in situ that are associated with non-rRNA analytes. In some embodiments, performing RCA of the rRNA-targeting probes can improve resolution and/or reduce optical crowding of any suitable signal detected from a non-rRNA analyte in a cell and/or tissue. For example, performing RCA of rRNA-targeting probes can improve detection of a signal generated using any suitable detection probe or probe set, such as any probe described herein, including an antibody or a nucleic acid probe. In some embodiments, performing RCA of rRNA-targeting probes improves detection of a protein (e.g. via immunofluorescence).

In some aspects, the expansion-microscopy-like effect achieved by performing RCA of rRNA-targeting probes can provide the advantages of improving resolution and/or reducing optical crowding of any suitable optical signal detected in a sample. Thus, in some embodiments, in the methods described herein, any suitable detection probe or probe set can be used to bind any suitable target analyte. The detection probe or probe set can be a nucleic acid probe that hybridizes to a target nucleic acid, but the detection probe or probe set need not be limited to a nucleic acid probe that hybridizes to a target nucleic acid. In some embodiments, the detection probe or probe set can be any suitable binding moiety. For example, in some embodiments, the detection probe or probe set can comprise an antibody, such as an antibody comprising a detectable label (e.g. fluorophore) that is used to generate an optical signal. In some embodiments, the method comprises: (a) contacting the biological sample with a detection probe or probe set, wherein the detection probe or probe set binds to a target analyte in the biological sample. In some embodiments, the method further comprises: (b) contacting the biological sample with a ribosomal RNA (rRNA)-targeting probe or probe set, wherein the rRNA-targeting probe or probe set hybridizes to a ribosomal RNA (rRNA) target sequence in the biological sample, and wherein the rRNA-targeting probe is a circular probe or the rRNA-targeting probe or probe set is a circularizable probe or probe set and the method comprises circularizing the rRNA-targeting probe or probe set; (c) performing rolling circle amplification (RCA) of the rRNA-targeting probe or probe set to generate a non-detection rolling circle amplification product (RCP) in the biological sample; and (d) imaging the biological sample to detect an optical signal associated with the detection probe or probe set or a product thereof, without detecting an optical signal associated with the non-detection RCP. In some embodiments, provided herein is a method of detecting a target analyte in a cell, comprising: (a) using a circular or circularizable ribosomal RNA (rRNA)-targeting probe or probe set to generate a plurality of non-detection rolling circle amplification products (RCPs) in the cell, thereby increasing the volume of the cell; and (b) detecting an optical signal associated with the target analyte. In some embodiments, the method does not comprise detecting an optical signal associated with the non-detection RCP. In some embodiments, the method comprises detecting a plurality of optical signals associated with one or more target analytes at a plurality of locations in the cell. In some embodiments, increasing the volume of the cell reduces optical crowding of the plurality of optical signals.

In some embodiments, performing rolling circle amplification of an rRNA-targeting probe or probe set in the biological sample results in physical expansion of the biological sample (e.g., physical expansion of a cell, cells, or tissue section), optionally wherein the sample is embedded in a matrix. In some embodiments, the RCA of an rRNA-targeting probe or probe set comprises RCA of at least 1,000, at least 2,000, at least 5,000, at least 10,000, at least 12,000, or at least 15,000 molecules of the circular or circularized rRNA-targeting probe. In some embodiments, the expansion is isometric expansion. In some embodiments, the expansion (e.g. isometric expansion) of the sample by RCA of a circular or circularized rRNA-targeting probe or probe set can increase the spatial resolution of the subsequent analysis of the sample. The increased resolution in spatial profiling can be determined by comparison of an isometrically expanded sample (e.g., a sample after performing RCA using circular or circularized rRNA-targeting probe or probe set) with a sample that has not been isometrically expanded. In some embodiments, the sample that has not been isometrically expanded is a sample that was not contacted with a circular or circularizable rRNA-targeting probe or probe set. In some embodiments, a biological sample is isometrically expanded using RCA of a circular or circularized rRNA-targeting probe or probe set to a size at least 1.1×, 1.2×, 1.3×, 1.4×, 1.5×, 1.6×, 1.7×, 1.8×, 1.9×, or 2.0× its non-expanded size. In some embodiments, the sample is isometrically expanded to at least 1.1× of its non-expanded size. In some embodiments, the sample is isometrically expanded to at least 1.5× of its non-expanded size. In some embodiments, a biological sample is isometrically expanded using RCA of a circular or circularized rRNA-targeting probe or probe set to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size. In some embodiments, a biological sample is expanded using RCA of a circular or circularized rRNA-targeting probe or probe set to a size at least 1.1×, 1.2×, 1.3×, 1.4×, 1.5×, 1.6×, 1.7×, 1.8×, 1.9×, or 2.0× its non-expanded size. In some embodiments, the sample is expanded to at least 1.1× of its non-expanded size. In some embodiments, the sample is expanded to at least 1.5× of its non-expanded size. In some embodiments, a biological sample is expanded using RCA of a circular or circularized rRNA-targeting probe or probe set to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is expanded to at least 2× and less than 20× of its non-expanded size. In some embodiments, the size of the sample refers to any suitable measure of the sample. In some embodiments, the size of the sample refers to the volume of the sample. In some embodiments, the size of the sample refers to the area of a sample. In some embodiments, the size of the sample refers to the area of a cross-section of the sample, such as an area in a tissue section, the area of a cell, or a portion thereof.

In some embodiments, performing RCA of a circular or circularized rRNA-targeting probe in the biological sample increases sensitivity for detection of a different target nucleic acid molecule by detecting a detection probe or probe set hybridized to the target nucleic acid molecule, or detecting an amplification product of the detection probe or probe set (referred to as a detection RCP). The present disclosure provides data demonstrating improved sensitivity for detecting target nucleic acids (e.g., mRNAs) after RCA of a circularized rRNA-targeting probe in the biological sample. For example, in some embodiments, the number of detected target nucleic acids (total number of detected objects) is increased in the biological sample. In some embodiments, performing RCA of a circular or circularized rRNA-targeting probe in the biological sample increases sensitivity for detection of a different target analyte by detecting a detection probe or probe set that binds to the target analyte.

### II. Methods for Analyzing a Biological Sample

In some aspects, provided herein are methods for analyzing analytes such as nucleic acids (e.g., transcripts representing the whole transcriptome or a portion thereof) in a biological sample (e.g., a tissue sample or cell). The methods provided herein comprise RCA methods and probes for improving spatial analysis. In some embodiments, the probes for RCA comprise a detection probe or probe set, wherein the detection probe or probe set hybridizes to a target nucleic acid in the biological sample, and a mixture of a circularizable and non-circularizable rRNA-targeting probe or probe set, which hybridizes to a ribosomal RNA (rRNA) target sequence in the biological sample. In some embodiments, RCA with a mixture of a circularizable and non-circularizable rRNA-targeting probe or probe set, which hybridizes to a ribosomal RNA (rRNA) target sequence in the biological sample is used to achieve the cellular expansion to mitigate physical and optical crowding in order to improve sensitivity and resolution of the target analyte, e.g. target nucleic acid, detection.

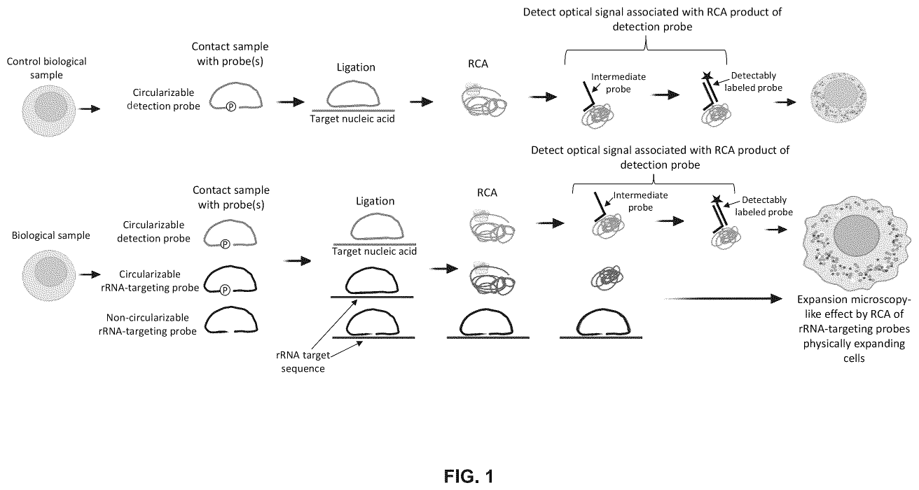

FIG. 1 shows an example workflow of a method of analyzing a biological sample using generation of RCPs from rRNA-targeting probes to provide an expansion microscopy-like effect. As shown in FIG. 1, the biological sample can be contacted with a circularizable rRNA-targeting probe and a non-circularizable rRNA-targeting probe. The sample can also be contacted with a detection probe or probe set, which can be a circularizable detection probe or probe set. In some embodiments, the sample is contacted with a probe mixture comprising the circularizable rRNA-targeting probe, the non-circularizable rRNA-targeting probe, and the circularizable detection probe. In some embodiments, the probes are contacted with the biological sample sequentially, in any order. The circularizable probes that hybridize to their respective target sequences are circularized by ligation. As shown in the bottom panel of FIG. 1, some of the rRNA in the biological sample may be hybridized by the circularizable rRNA-targeting probe, which can then be ligated and amplified by RCA. Some of the rRNA in the biological sample may be hybridized by the non-circularizable rRNA-targeting probe, which is not circularized and cannot serve as a template for RCA. Thus, the ratio between the circularizable rRNA-targeting probe and the non-circularizable rRNA-targeting probe can be used to control the number of RCA products produced from rRNA-targeting probes in the biological sample.

As shown in the bottom panel of FIG. 1, the circularized rRNA-targeting probes and the circularized detection probes are amplified by RCA. The circularized detection probes can then be detected using any of the methods for detecting or analyzing nucleic acid sequences disclosed herein (e.g., binding a detectably labeled probe to the RCA product of the detection probe (the detection RCP), either directly or via hybridization to one or more intermediate probes that bind to the RCA product). In some embodiments, the RCA of the circularized rRNA-targeting probe results in a physical expansion of the cells in the biological sample and/or improved optical resolution of an optically detectable signal associated with the detection RCP.

In some cases, an expanded cell or tissue area, volume, or thickness, or an improved optical resolution, sensitivity (e.g., number of detected target nucleic acids/detection RCPs), or other improved characteristic herein is described in relation to a control sample or control method. A control sample/method can be a sample on which the method is performed under substantially similar conditions, but without RCA of a circular or circularized circularizable rRNA-targeting probe. For example, the top panel of FIG. 1 illustrates a control biological sample that is contacted with the circularizable detection probe (e.g., the same circularizable detection probe used in the method with a circularizable rRNA-targeting probe shown in the bottom panel). The circularizable detection probe is ligated and amplified by RCA, and the resulting RCA product is detected in the biological sample. As shown in FIG. 1, RCA of the circularized rRNA-targeting probe can result in physical expansion of a cell or tissue section in a biological sample, compared to the size of the control sample.

In some aspects, the methods provided herein comprise forming a hybridization product in a biological sample (e.g., a hybridization product formed between a probe such as a detection probe, rRNA-targeting probe, or non-circularizable probe, or any of the intermediate and/or detectably labeled probes herein, and a corresponding target sequence). In some embodiments, a hybridization product comprising the pairing of substantially complementary or complementary nucleic acid sequences within two different molecules can be analyzed. For example, hybridization of an endogenous analyte or a labeling agent (e.g., a reporter oligonucleotide attached to an antibody) with a detection probe or probe set can be analyzed. In some embodiments, a hybridization product between a detection probe or probes and a target nucleic acid is analyzed by binding detectably labeled probes (e.g., fluorescently labeled probes) directly or indirectly to the detection probe or probes or to an amplification product of the detection probe. Pairing for hybridization can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

In any of the embodiments herein comprising RCA, the method can comprise contacting the biological sample with one or more primers to perform the RCA. A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. Primers can vary in length. For example, primers can be about 6 bases to about 120 bases. For example, primers can include up to about 25 bases. A primer, may in some cases, refer to a primer binding sequence.

A. Generation of RCPs from rRNA-Targeting Probes or Probe Sets

In some embodiments, a method provided herein comprises contacting the biological sample with a ribosomal RNA (rRNA)-targeting probe or probe set, wherein the rRNA-targeting probe or probe set hybridizes to a ribosomal RNA (rRNA) target sequence in the biological sample, and wherein the rRNA-targeting probe is a circular probe or the rRNA-targeting probe or probe set is a circularizable probe or probe set and the method comprises circularizing the rRNA-targeting probe or probe set; and performing rolling circle amplification of the rRNA-targeting probe or probe set to generate a non-detection rolling circle amplification product (RCP) in the biological sample. The non-detection RCP can be detected (e.g., wherein the non-detection RCP comprises a barcode sequence identifying the rRNA-targeting probe or probe set), or the non-detection RCP may not be detected. In some embodiments, the non-detection RCP is not detected. In some cases, the method comprises imaging the sample to analyze a barcode sequence in a detection probe or probe set that hybridizes to a target nucleic acid other than the target rRNA, or a RCA product of the detection probe or probe set (e.g. a detection RCP). In some embodiments, the method does not comprise detecting the rRNA-targeting probe or probe set in an imaging step for analyzing a barcode sequence of the detection probe or probe set or detection RCP.

In some embodiments, the biological sample is contacted with a composition comprising no more than any one of 5 nM, 4 nM, 3 nM, 2 nM, 1 nM, 0.5 nM, 0.25 nM, 0.1 nM, or 0.05 nM of a circular or circularizable rRNA-targeting probe or probe set. In some embodiments, the biological sample is contacted with a composition having between 0.05 nM and 5 nM, between 0.05 nM and 1 nM, or between 0.05 nM and 0.5 nM of a circular or circularizable rRNA-targeting probe or probe set. In some embodiments, the biological sample is contacted with a composition comprising no more than any one of 5 nM, 4 nM, 3 nM, 2 nM, 1 nM, 0.5 nM, 0.25 nM, 0.1 nM, or 0.05 nM of a circularizable rRNA-targeting probe. In some embodiments, the biological sample is contacted with a composition having between 0.05 nM and 5 nM, between 0.05 nM and 1 nM, or between 0.05 nM and 0.5 nM of a circularizable rRNA-targeting probe. In some embodiments, the biological sample is contacted with a composition having about 0.5 nM of a circularizable rRNA-targeting probe.

In some embodiments, the rRNA-targeting probe is a circular probe or a circularizable probe or probe set. In some embodiments, the method comprises circularizing the circularizable rRNA probe or probe set. In some embodiments, rRNA-targeting probe or probe set is circularized by ligation selected from the group consisting of enzymatic ligation, chemical ligation, template dependent ligation, and/or template independent ligation. In some embodiments, the rRNA-targeting probe is circularized by enzymatic ligation, wherein the enzymatic ligation comprises using a ligase having an RNA-templated DNA ligase activity and/or an RNA-templated RNA ligase activity. In some embodiments, the enzymatic ligation comprises using a ligase selected from the group consisting of a Chlorella virus DNA ligase (PBCV DNA ligase), a T4 RNA ligase, a T4 DNA ligase, and a single-stranded DNA (ssDNA) ligase.

In some embodiments, the ligation to circularize the circularizable rRNA-targeting probe is templated by the target rRNA. For example, the 5′ and the 3′ end of the circularizable rRNA-targeting probe can be brought into proximity with each other upon hybridization to the target rRNA. In an example, the rRNA-targeting probe is a padlock probe. In some embodiments, the ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). In some embodiments, the ligation is performed without gap-filling. In some embodiments, the circularizable rRNA-targeting probe or probe set is circularized using a splint. In some cases, the circularizable rRNA-targeting probe or probe set is a circularizable rRNA-targeting probe set comprising a first rRNA-targeting probe and a second rRNA-targeting probe that are circularized by at least two ligations of the 5′ end of the first rRNA-targeting probe to the 3′ end of the second rRNA-targeting probe, and of the 3′ end of the first rRNA-targeting probe to the 5′ end of the second rRNA-targeting probe.

In some embodiments, upon addition of a DNA polymerase in the presence of appropriate dNTP precursors and other cofactors, a primer is elongated to produce multiple copies of the circular or circularized rRNA-targeting probe or probe set. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Examples of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some embodiments, the non-detection RCPs are generated using the circular or circularized rRNA-targeting probe or probe set as a template, and using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In some aspects, the non-detection RCPs (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

In some embodiments, the method herein comprises performing RCA of molecules of an rRNA-targeting probe or probe set in a biological sample. In some embodiments, the method comprises performing RCA of a circular or circularized rRNA-targeting probe or probe set targeting highly abundance rRNA transcripts to generate rRNA RCPs in a quantity sufficient for physical cell area expansion. In some embodiments, RCA of molecules of an rRNA-targeting probe or probe set in a biological sample results in cell area expansion. In some embodiments, RCA of molecules of an rRNA-targeting probe or probe set in a biological sample results in cell volume expansion. In some embodiments, RCA of molecules of an rRNA-targeting probe or probe set in a biological sample results in increased thickness of a tissue section.

rRNA is the primary component of ribosomes and are encoded by essential housekeeping genes found in all organisms. rRNAs are non-coding RNA transcripts that make up the predominant form of RNA found in most cells. rRNA transcript levels are highly abundant in comparison to other RNA transcripts levels in the cell. In some embodiments, the target rRNA accounts for at least 10%, at least 15%, at least 20%, at least 25%, at least 30%, or at least 50% of total cellular RNA in a biological sample. In some embodiments, the target rRNA accounts for at least 20% of total cellular RNA in a biological sample. In some embodiments, the target rRNA accounts for between about 10% and about 80%, between about 15% and about 70%, or between about 20% and about 70% of total cellular RNA in the biological sample.

Eukaryotic cells have at least four types of rRNA: 5S, 5.8S, 18S and 28S rRNAs. The 5S, 5.8S and 28S rRNAs are found within the large ribosomal subunit, while the 18S rRNA is located in the small ribosomal subunit. In some embodiments, the rRNA-targeting probe or probe set hybridizes to 5S, 5.8S, 18S, or 28S rRNAs in the biological sample, or to any combination thereof. In some embodiments, the rRNA-targeting probe or probe set hybridizes to 5S rRNA. In some embodiments, the rRNA-targeting probe or probe set hybridizes to 5.8S rRNA. In some embodiments, the rRNA-targeting probe or probe set hybridizes to 18S rRNA. In some embodiments, the rRNA-targeting probe or probe set hybridizes to 28S rRNA. In some embodiments, the rRNA-targeting probe or probe set hybridizes to 18S rRNAs in the biological sample. In some embodiments, provided herein are a plurality of rRNA targeting probes or probe sets for targeting a plurality of target rRNAs. In addition, the mitochondria and chloroplasts in eukaryotes also have their own rRNAs (for instance, the 12S and 16S mitochondrial rRNAs in mammals), which are grossly similar to those in the prokaryotes. In some embodiments, the rRNA-targeting probe or probe set hybridizes to 12S or 16S rRNAs in the biological sample. In some embodiments, the biological sample is a eukaryotic sample, and the rRNA is selected from the group consisting of 18S rRNA, 28S rRNA, and 5.8S rRNA. In some embodiments, the rRNA is 18S rRNA.

In prokaryotes such as bacteria and archaea, there are at least three types of rRNA: 16S, 23S, and 5S rRNA. In some embodiments, the rRNA-targeting probe or probe set hybridizes to 16S, 23S, or 5S rRNAs in the biological sample, or any combination thereof. In some embodiments, the rRNA-targeting probe or probe set hybridizes to 16S rRNA. In some embodiments, the rRNA-targeting probe or probe set hybridizes to 23S rRNA. In some embodiments, the rRNA-targeting probe or probe set hybridizes to 5S rRNA. In some embodiments, the biological sample is a prokaryotic sample, and the rRNA is 23S or 16S rRNA.

In some embodiments, RCPs are produced by RCA in an abundance sufficient to physically expand the cell. In some embodiments, the expansion is isometric expansion. Not only are rRNAs relatively abundant compared to other transcripts in the cell, they are remarkably abundant in absolute numbers. For example, a single actively replicating eukaryotic cell may contain as many as 10 million ribosomes, which are comprised of rRNA, while prokaryotes such as Escherichia coli can have as many as 15,000 ribosomes. In some embodiments, at least 1,000 molecules of the rRNA-targeting probe hybridize to rRNAs per cell in the biological sample.

In some embodiments, the rolling circle amplification is performed for at least or at least about any of 30 minutes, 1 hour, 1.5 hours, 2 hours, 2.5 hours, 3 hours, or overnight. In some embodiments, the rolling circle amplification is performed for about any of 30 minutes, 40 minutes, 45 minutes, 50 minutes, 60 minutes, 70 minutes, 80 minutes, 90 minutes, 120 minutes, or 360 minutes, or any range that is formed from any two of those values as endpoints.

In some embodiments, the rolling circle amplification is performed at between about 4° C. and about 65° C., between about 18° C. and about 50° C., between about 30° C. and about 45° C., or between about 30° C. and about 40° C. In some embodiments, the rolling circle amplification is performed at a temperature of about 20° C., about 25° C., about 30° C., about 35° C., about 40° C., about 45° C., about 50° C., about 55° C., or about 60° C., or any range that is formed from any two of those values as endpoints. In some embodiments, the rolling circle amplification is performed at a temperature of about 37° C.

In some embodiments, performing rolling circle amplification of the rRNA-targeting probe comprises generating between about 5,000 to about 50,000 RCPs per cell using the rRNA targeting probe as a template, about 7,500 to about 25,000 RCPs per cell, or between about 10,000 to 15,000 RCPs per cell from the molecules of the rRNA targeting probe. In some embodiments, performing rolling circle amplification of the rRNA-targeting probe comprises generating between about 10,000 to 15,000 RCPs per cell from the molecules of the rRNA targeting probe. In some embodiments, performing rolling circle amplification of the rRNA-targeting probe comprises generating between about 12,500 RCPs per cell and about 15,000 RCPs per cell from the molecules of the rRNA targeting probe.

In some embodiments, the rRNA-associated RCPs (e.g. non-detection RCPs) are not detected. In some embodiments, the production of rRNA-associated RCPs is used to mitigate physical and/or optical crowding in order to improve spatial analysis of the target analyte (e.g. target nucleic acid) to which the detection probe or probe set binds and/or hybridizes. In some embodiments, the RCPs produced from an rRNA-targeting probe in the biological sample are not detected using a fluorescently labeled probe. In some embodiments, an RCP generated by RCA using the circular or circularized detection probe or a circularized detection probe set as a template is detected in the biological sample (e.g., using a fluorescently labeled probe) and the RCPs produced from an rRNA-targeting probe in the biological sample are not detected using a fluorescently labeled probe.

### B. Non-Circularizable Probes or Probe Sets

In some embodiments, generation of RCPs by performing rolling circle amplification of the rRNA-targeting probe is tunable. In some embodiments, the tuning of RCA comprises contacting the biological sample with a circularizable probe or probe set and a non-circularizable probe or probe set. In some embodiments, the method described herein comprises contacting the biological sample with a non-circularizable probe or probe set, wherein the non-circularizable probe or probe set comprises a hybridization region complementary to the rRNA target sequence or to a portion of the rRNA target sequence. In some embodiments, the non-circularizable probe or probe set comprises a sequence that is at least 95%, at least 96%, at least 97%, at least 98%, at least 99%, or 100% identical to the region of the ribosomal RNA (rRNA)-targeting probe or probe set that is complementary to the target rRNA.

In some embodiments, the non-circularizable probe or probe set is a linear probe. In some embodiments, the linear probe hybridizes to the rRNA such that the 3′ end of the linear probe is not adjacent to and/or is not juxtaposed to the 5′ end of the linear probe. For example, the entire length of the linear probe can be hybridized to the rRNA, such that an extension of the hybridized 3′ end of the linear probe would not proceed in the direction of the hybridized 5′ end of the linear probe (e.g. in contrast to the configuration of a padlock probe). In some embodiments, the 5′ end of the non-circularizable probe or probe set lacks a 5′ phosphate, thereby preventing ligation to the 3′ end of the probe. To generate RCPs, the 5′ phosphate is required for ligation to the 3′-end in order to initiate RCA. Removal of the 5′ phosphate can be accomplished through enzymatic means, such as treatment with an alkaline phosphatase. In some embodiments, the non-circularizable probe or probe set has been treated with an alkaline phosphatase prior to contacting the biological sample. In some embodiments, the linear probe also hybridizes to the rRNA target. In some embodiments, the circularizable rRNA-targeting probe and non-circularizable rRNA-targeting probe compete for binding of the rRNA target. In some embodiments, the competition between the circularizable and non-circularizable probe for hybridization reduces the number of RCA products.

In some embodiments, productive RCA of rRNA-targeting probes is tuned by varying the ratios of circularizable rRNA-targeting probes and non-circularizable rRNA-targeting probes in the probe mixture. In some embodiments, the ratio of the rRNA-targeting probe or probe set to the non-circularizable probe or probe set is between about 1:5 and about 1:20, or between about 1:8 and 1:12. In some embodiments, the circular or circularizable rRNA-targeting probe or probe set is contacted with the biological sample at a concentration of between about 0.05 and about 5 nM, between about 0.5 nM and about 2.5 nM, between about 0.5 nM and about 1 nM, or between about 0.1 nM and about 1 nM. In some embodiments, the circular or circularizable rRNA-targeting probe or probe set is contacted with the biological sample at a concentration of about 0.5 nM. In some embodiments, the non-circularizable probe or probe set is contacted with the biological sample at a concentration of between about 0.05 and about 5 nM, between about 0.5 and about 2.5 nM, between about 0.5 and about 1 nM, or between about 0.1 and about 1 nM. In some embodiments, the non-circularizable probe or probe set is contacted with the biological sample at a concentration of about 4.5 nM. For example, the probe mixture comprises 0.5 nM of circularizable rRNA targeting probes and 4.5 nM of non-circularizable rRNA targeting probes (both targeting the same rRNA). In some aspects, the probe mixture is tuned to achieve an increase in sensitivity for detection of the target nucleic acids (e.g., of detection RCPs) without resulting in a disrupted nucleus morphology from the expansion.

In some embodiments, the rRNA-targeting probe or probe set and the non-circularizable probe or probe set are contacted with the biological sample simultaneously. In some embodiments, the rRNA-targeting probe or probe set is a circularizable probe or probe set, and the method comprises circularizing the rRNA-targeting probe or probe set by ligation (e.g., in an enzymatic ligation that depends on hybridization of the rRNA-targeting probe or probe set to the rRNA). In some embodiments, the non-circularizable probe or probe set is not competent for ligation by an enzyme that circularizes the rRNA-targeting probe or probe set. In some embodiments, the enzymatic ligation of the rRNA-targeting probe or probe set comprises using a ligase having an RNA-templated DNA ligase activity and/or an RNA-templated RNA ligase activity, and the ligase cannot circularize the non-circularizable probe or probe set under the conditions wherein the non-circularizable probe or probe set is hybridized to the rRNA. In some embodiments, the non-circularizable probe or probe set hybridizes to the rRNA such that a 5′ and a 3′ end of the non-circularizable probe or probe set are not juxtaposed for ligation, with or without gap-filling. In some embodiments, the non-circularizable probe or probe set comprises a 5′ phosphate and a 3′ OH, but the ends of the non-circularizable probe or probe set are not juxtaposed for ligation upon hybridization to the rRNA.

### C. Detection Probes or Probe Sets

In some embodiments, provided herein are methods and compositions for analyzing one or more analytes (e.g., endogenous analytes) in a biological sample. In some embodiments, the one or more analytes are analyzed at their relative spatial positions in a biological sample (e.g., wherein the biological sample is physically expanded by rolling circle amplification of circular or circularized rRNA-targeting probes, and the relative spatial positions of the one or more analytes are maintained in the expanded sample).

The detection probe or probe set can be any probe or probe set suitable for detecting a target analyte, such as a protein or a target nucleic acid. The detection probe or probe set can be any probe or probe set suitable for detecting a target nucleic acid (e.g., an endogenous nucleic acid analyte or a nucleic acid that is part of a labeling agent, such as a reporter oligonucleotide conjugated to an antibody or antigen binding fragment for detecting a non-nucleic acid analyte). Various detection probes and probe sets can be hybridized to an endogenous analyte and/or a labeling agent and each detection probe or probe set may comprise one or more barcode sequences. Examples of barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

In some embodiments, the target nucleic acid is a ligation product of an endogenous analyte and/or a labeling agent that is analyzed using a detection probe or probe set provided herein. In some embodiments, the ligation product is formed between two or more endogenous analytes. In some embodiments, the ligation product is formed between two or more labeling agents. In some embodiments, the ligation product is an intramolecular ligation of an endogenous analyte.

In some embodiments, the detection probe or probe set comprises a target recognition sequence complementary to a target nucleic acid in the biological sample. In some embodiments, the detection probe or probe set comprises a plurality of detection probes that hybridize to a plurality of target sequences in a target nucleic acid. In some embodiments, the detection probe or probe set comprises at least 10, 20, 30, or 40 probes that hybridize to target sequences tiling a target nucleic acid (e.g., an RNA transcript). In some embodiments, the detection probe or probe set comprises between about 15 and about 40 probes that hybridize to different target sequences tiling the RNA transcript. In some embodiments, the detection probe or probe set can be any suitable mRNA-targeting probe or probe set.

In some embodiments, the biological sample is contacted with a composition comprising no more than any one of 10 nM, 5 nM, 4 nM, 3 nM, 2 nM, 1 nM, 0.5 nM, 0.25 nM, 0.1 nM, or 0.05 nM of a circular or circularizable detection probe or probe set. In some embodiments, the biological sample is contacted with a composition having between 0.05 nM and 10 nM, between 0.05 nM and 1 nM, between 0.05 nM and 5 nM, between 3 nM and 8 nM, between 4 nM and 6 nM, or between 4.5 nM and 5 nM of a circular or circularizable mRNA-targeting detection probe or probe set. In some embodiments, the biological sample is contacted with a composition comprising no more than any one of 10 nM, 5 nM, 4 nM, 3 nM, 2 nM, 1 nM, 0.5 nM, 0.25 nM, 0.1 nM, or 0.05 nM of a circularizable mRNA-targeting detection probe or probe set. In some embodiments, the biological sample is contacted with a composition having between 0.05 nM and 5 nM, between 0.05 nM and 1 nM, between 0.05 nM and 5 nM, between 3 nM and 8 nM, between 4 nM and 6 nM, or between 4.5 nM and 5 nM of a circularizable mRNA-targeting detection probe or probe set.

In some embodiments, the detection probe or probe set is circularized by ligation. In some embodiments, the ligation product is an intramolecular ligation product or an intermolecular ligation product, for example, the ligation product can be generated by the circularization of a circularizable probe or probe set upon hybridization to a target sequence in the target nucleic acid. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some embodiments, the detection probe or probe set is a probe or probe set capable of DNA-templated ligation, such as from a cDNA molecule. See, e.g., U.S. Pat. No. 8,551,710, which is hereby incorporated by reference in its entirety. In some embodiments, the detection probe or probe set comprises one or more ribonucleotides and/or a cleavable 5′ flap for RNA-templated ligation. See, e.g., U.S. Pat. Pub. 2020/0224244 which is hereby incorporated by reference in its entirety. In some embodiments, detection probe or probe set is a SNAIL probe set. See, e.g., U.S. Pat. Pub. 20190055594, which is hereby incorporated by reference in its entirety. In some embodiments, the detection probe or probe set provided herein is for a multiplexed proximity ligation assay. See, e.g., U.S. Pat. Pub. 20140194311 which is hereby incorporated by reference in its entirety. In some embodiments, detection probe or probe set is capable of proximity ligation, for instance in a proximity ligation assay for RNA (e.g., PLAYR) probe set. See, e.g., U.S. Pat. Pub. 20160108458, which is hereby incorporated by reference in its entirety. In some embodiments, a detection probe herein is a circular probe that is indirectly bound to the target nucleic acid via hybridization to a primary probe that hybridizes to the target nucleic acid. In some embodiments, the circularized detection probe or probe set is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation of any of the ligatable probes described herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the detection probe or probe set produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). The two or more nucleic acid sequences can be brought into proximity to each other by hybridization to a target nucleic acid or a separate splint nucleic acid. In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

In some embodiments, the method comprises contacting the biological sample with a primer complementary to a primer-binding sequence in the detection probe or probe set. In some embodiments, the primer binding sequence is common among a plurality of different detection probes or probe sets for different target nucleic acids. In some embodiments, the primer binding sequence is the same in one or more detection probes or probe sets and in one or more rRNA-targeting probes or probe sets. In some embodiments, the primer further comprises a target-recognition sequence that hybridizes to a sequence of the target nucleic acid adjacent or in proximity to the sequence of the target nucleic acid that is hybridized by the detection probe or probe set. In some embodiments, the primer does not comprise a sequence that hybridizes specifically to the target nucleic acid.

In some embodiments, a product of an endogenous analyte and/or a labeling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C., or any range having any of the foregoing values as endpoints.

In some embodiments, upon addition of a DNA polymerase in the presence of appropriate dNTP precursors and other cofactors, a primer is elongated to produce multiple copies of the circular or circularized detection probe or probe set. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Examples of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the detection RCPs (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the detection RCP is generated as a proxy, or a marker, for the analyte. In some embodiments, different analytes are detected in situ in one or more cells using a RCA-based detection system, e.g., where the signal is provided by generating a detection RCA product from a circular RCA template which is provided or generated in the assay, and the detection RCA product is detected to detect the corresponding analyte. The detection RCA product may thus be regarded as a reporter which is detected to detect the target analyte. However, the detection RCA template (the detection probe or probe set) may also be regarded as a reporter for the target analyte; the RCA product is generated based on the RCA template, and comprises complementary copies of the RCA template. The RCA template determines the signal which is detected, and is thus indicative of the target analyte.

In some embodiments, a detection probe or probe set provided herein comprises one or more barcode(s), e.g., at least two, three, four, five, six, seven, eight, nine, ten, or more barcodes. Barcodes can spatially-resolve molecular components found in biological samples, for example, within a cell or a tissue sample. A barcode sequence or complement thereof can be attached to an target nucleic acid by hybridization of the detection probe or probe set to the target nucleic in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some embodiments, a rolling circle amplification product is generated using a circular or circularized detection probe or probe set as a template for amplification, thereby providing the complement of a barcode sequence in the RCA product formed from the detection probe or probe set comprising the barcode sequence. The complement of the barcode sequence in the RCA product can then be detected using any of the methods described in Section II.D.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including target nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

### D. Detection and Analysis

In some aspects, the provided methods involve analyzing, e.g., detecting or determining, one or more sequences present in the detection probes or probe sets or products thereof (e.g., rolling circle amplification products thereof). In some embodiments, the detecting is performed at one or more locations in the biological sample. In some embodiments, the detecting is performed at one or more locations in the biological sample after RCA of the rRNA-targeting probes in the biological sample. In some embodiments, the locations are the locations of RNA transcripts in the biological sample. In some embodiments, the locations are the locations of RNA transcripts or their corresponding locations after performing RCA of rRNA-targeting probes in the biological sample. In some embodiments, the RCA using rRNA-targeting probes or probe sets in the biological sample physically expands the cell(s) or tissue of biological sample. In some embodiments, the relative spatiality of target analytes, such as target nucleic acids (e.g., RNA transcripts) in the biological sample is preserved following expansion of the biological sample by RCA of rRNA-targeting probes. For example, detected target nucleic acids (or detection probes or probe sets or detection RCPs that serve as a proxy for target nucleic acids in the biological sample) can be accurately assigned to distinct cells in the biological sample. In some embodiments, the locations are the locations at which the detection probes or probe sets hybridize to the target nucleic acids (e.g., RNA transcripts or labeling agents) in the biological sample, and are optionally ligated and amplified by rolling circle amplification.

In some embodiments, the methods provided herein comprise analyzing a barcode sequence or complement thereof in the detection probe or probe set or in a product of the detection probe or probe set (e.g., a detection RCP) at a location in the biological sample or a matrix embedding the biological sample. In some cases, the barcode sequence or complement thereof is analyzed after RCA to form RCPs from molecules of a circular or circularized rRNA-targeting probe or probe set in the biological sample. In some embodiments, the RCA to form RCPs from molecules of a circular or circularized rRNA-targeting probe or probe set in the biological sample improves the resolution of the detection RCPs during a subsequent imaging step to detect the detection RCPs. In some embodiments, the RCA to form RCPs from molecules of a circular or circularized rRNA-targeting probe or probe set in the biological sample improves sensitivity for detection of the detection RCPs in subsequent imaging or sequence analysis. In some cases, the barcode sequence or complement thereof is analyzed by sequential hybridization, sequencing by hybridization, sequencing by ligation, sequencing by synthesis, sequencing by binding, or a combination thereof.

In some embodiments, analyzing the barcode sequence or complement thereof comprises hybridizing a detectably labeled probe to the barcode sequence or complement thereof, or a subunit of the barcode sequence or complement thereof, and detecting the detectably labeled probe. In some instances, the detectably labeled probe comprises a fluorescent label, and after detecting the detectably labeled probe, the method comprises removing the detectably labeled probe, quenching the fluorescent label, and/or cleaving and removing the fluorescent label. Analyzing the barcode sequence or complement thereof can comprise hybridizing a second detectably labeled probe to the barcode sequence or complement thereof, or a subunit of the barcode sequence or complement thereof, and detecting the second detectably labeled probe. In some embodiments, the barcode sequence or complement thereof is assigned a series of signal codes that identifies the barcode sequence or complement thereof, and detecting the barcode sequence or complement thereof comprises decoding the barcode sequence or complement thereof by detecting the corresponding series of signal codes detected from sequential hybridization, detection, and removal, quenching, and/or cleavage of detectably labeled probes. In some embodiments, the series of signal codes is a fluorophore signature assigned to the corresponding barcode sequences or complements thereof.

In some embodiments, analyzing the barcode sequence or complement thereof comprises hybridizing an intermediate probe to the barcode sequence or complement thereof, or a subunit of the barcode sequence or complement thereof, hybridizing a detectably labeled probe to the intermediate probe, and detecting the detectably labeled probe. In some instances, detecting the barcode sequence or complement thereof comprises contacting the biological sample with a universal pool of detectably labeled probes and a first pool of intermediate probes, wherein an intermediate probe of the first pool of intermediate probes comprises a hybridization region complementary to the barcode sequence or complement thereof and a reporter region complementary to a detectably labeled probe of the universal pool of detectably labeled probes. The method can then comprise detecting a complex formed between the barcode sequence or complement thereof, the intermediate probe of the first pool of intermediate probes, and the detectably labeled probe; and removing the intermediate probe of the first pool of intermediate probes and the detectably labeled probe. In some embodiments, detecting the barcode sequence or complement thereof further comprises: contacting the biological sample with the universal pool of detectably labeled probes and a second pool of intermediate probes, wherein an intermediate probe of the second pool of intermediate probes comprises a hybridization region complementary to the barcode sequence or complement thereof and a reporter region complementary to a detectably labeled probe of the universal pool of detectably labeled probes; and detecting a complex formed between the barcode sequence or complement thereof, the intermediate probe of the second pool of intermediate probes, and the detectably labeled probe. In some embodiments, the barcode sequence or complement thereof is assigned a series of signal codes that identifies the barcode sequence or complement thereof, and detecting the barcode sequence or complement thereof comprises decoding the barcode sequence or complement thereof by detecting the corresponding series of signal codes detected from sequential hybridization, detection, and removal of sequential intermediate probes and the universal pool of detectably labeled probes. In some embodiments, the series of signal codes is a fluorophore signature assigned to the corresponding barcode sequences or complements thereof.

In some embodiments, detecting the one or more sequences present in the probes or probe sets (e.g., the detection probes or probe sets, or products thereof) in the biological sample is performed, and the detected sequences are compared to an expected set of detected sequences. In some embodiments, the expected set of sequences is based on the barcode sequences of the panels of detection probes or probe sets in the probe mixture and the known expression levels of the RNA transcripts in particular cells or regions of the biological sample.

In some embodiments, the detecting comprises a plurality of repeated cycles of hybridization and removal of probes (e.g., detectably labeled probes, or intermediate probes that bind to detectably labeled probes) to the primary probe or probe set hybridized to the target nucleic acid, or to a rolling circle amplification product generated from the probe or probe set hybridized to the target nucleic acid.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization assays, or sequencing by hybridization).

In some embodiments, the detecting can comprise binding an intermediate probe directly or indirectly to the detection probe or probe set, binding a detectably labeled probe directly or indirectly to a detection region of the intermediate probe, and detecting a signal associated with the detectably labeled probe. In some embodiments, the method comprises detecting a rolling circle amplification product (RCP) generated using a circular or circularized detection probe or probe set as a template. In some embodiments, the method comprises detecting a rolling circle amplification product (RCP) generated using a circular or circularized detection probe or probe set that binds to a primary probe or probe set as a template. In some embodiments, detecting the RCP comprises binding an intermediate probe directly or indirectly to the RCP, binding a detectably labeled probe directly or indirectly to a detection region of the intermediate probe, and detecting a signal associated with the detectably labeled probe. In some embodiments, the method can comprise performing one or more wash steps to remove unbound and/or nonspecifically bound intermediate probe molecules from the primary probes or the products of the primary probes.

In some embodiments, the detecting can comprise: detecting signals associated with detectably labeled probes that are hybridized to barcode regions or complements thereof in the detection probe or probe set or a product thereof (e.g., a detection RCP); and/or detecting signals associated with detectably labeled probes that are hybridized to intermediate probes which are in turn hybridized to the barcode regions or complements thereof in the detection probe or probe set. In some embodiments, the detectably labeled probes can be fluorescently labeled.

In some embodiments, the methods comprise detecting the sequence in all or a portion of a detection probe or probe set or a detection RCP, or detecting a sequence of the detection probe or probe set or detection RCP, such as one or more barcode sequences present in the detection probe or probe set or detection RCP. In some embodiments, the sequence of the detection RCP, or barcode thereof, is indicative of a sequence of the target nucleic acid to which the detection probe or probe set is hybridized. In some embodiments, the analysis and/or sequence determination comprises detecting a sequence in all or a portion of the nucleic acid concatemer and/or in situ hybridization to the detection RCP. In some embodiments, the detection step involves sequencing by hybridization, sequencing by ligation, sequencing by synthesis, sequencing by binding, and/or fluorescent in situ sequencing (FISSEQ), and/or hybridization-based in situ sequencing. In some embodiments, the detection step is by sequential fluorescent in situ hybridization (e.g., for combinatorial decoding of the barcode sequence or complement thereof).

In some embodiments, the detection or determination comprises hybridizing to the detection probe directly or indirectly a detectably-labeled probe (e.g., a probe labeled with a fluorophore, an isotope, a mass tag, or a combination thereof). In some embodiments, the detection or determination comprises imaging the detection probe or probe set or detection RCP hybridized to the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample. In some embodiments, the target nucleic acid is an mRNA in a tissue sample, and the detection or determination is performed when the target nucleic acid and/or the amplification product is in situ in the tissue sample, after RCA of the rRNA-targeting probes or probe sets in the biological sample. In some embodiments, the target nucleic acid is an mRNA in a tissue sample, and the detection or determination is performed when the target nucleic acid and/or the amplification product is in situ in the tissue sample, after physical expansion of the cell(s) and/or tissue in the biological sample by performing RCA of the rRNA-targeting probes or probe sets in the biological sample. In some embodiments, the target nucleic acid is an amplification product (e.g., a rolling circle amplification product).

In some embodiments, the optical signal associated with the detection probe or probe set or a product thereof is enhanced by signal amplification in the biological sample. In some embodiments, the signal amplification comprises hybridization chain reaction (HCR) of a circularizable probe that directly or indirectly to the target nucleic acid; linear oligonucleotide hybridization chain reaction (LO-HCR) directly or indirectly to the target nucleic acid; branched oligonucleotide hybridization chain reaction directly or indirectly to the target nucleic acid; primer exchange reaction (PER) directly or indirectly to the target nucleic acid, or any combination thereof.

In some instances, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. No. 7,632,641 (see also US 2006/0234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described (see, e.g., WO 2020/123742 incorporated herein by reference), and may be used in the methods herein.

In some instances, similar to HCR reactions that use hairpin monomers, linear oligo hybridization chain reaction (LO-HCR) can also be used for signal amplification. In some instances, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some instances, the first species and/or the second species may not comprise a hairpin structure. In some instances, the plurality of LO-HCR monomers may not comprise a metastable secondary structure. In some instances, the LO-HCR polymer may not comprise a branched structure. In some instances, performing the linear oligo hybridization chain reaction comprises contacting the target nucleic acid molecule with the initiator to provide the initiator hybridized to the target nucleic acid molecule. In any of the instances herein, the target nucleic acid molecule and/or the analyte can be an RCA product.

In some instances, detection of nucleic acids sequences in situ includes combination of the sequential decoding methods described herein with an assembly for branched signal amplification. In some instances, the assembly complex comprises an amplifier hybridized directly or indirectly (via one or more oligonucleotides) to a sequence of an oligonucleotide probe described herein. In some instances, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some instances, the amplifier repeating sequence is about 5-30 nucleotides, and is repeated N times in the amplifier. In some instances, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some instances, an oligonucleotide probe described herein can be associated with an amplified signal by a method that comprises signal amplification by performing a primer exchange reaction (PER). In various instances, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various instances, the strand displacing polymerase is Bst. In various instances, the catalytic hairpin includes a stopper which releases the strand displacing polymerase. In various instances, branch migration displaces the extended primer, which can then dissociate. In various instances, the primer undergoes repeated cycles to form a concatemer primer (see e.g., U.S. Pat. Pub. No. US20190106733, which is incorporated herein by reference, for exemplary molecules and PER reaction components).

In some instances, the disclosed methods may comprise the use of a branched DNA (bDNA) amplification approach to amplify signals. In branched DNA (bDNA) amplification, primary and secondary amplifier oligonucleotides, each containing multiple replicate binding sites, are assembled on, e.g., individual smFISH probes to form a branched structure which binds multiple copies of a fluorescently labeled probe (Xia, et al. (2019), “Multiplexed Detection of RNA Using MERFISH and Branched DNA Amplification”, Scientific Reports 9:7721). The degree of amplification in bDNA amplification is controlled by the design of the amplification reaction, i.e., the assembled bDNA structures cannot grow indefinitely even in the presence of excess reagents, which may be used to control spot size or limit the variability in brightness from molecule to molecule (Xia, et al. (2019), ibid.).

In some instances, the disclosed methods may comprise the use of a hybridization chain reaction (HCR) approach to amplify signals. In a hybridization chain reaction, two fluorescently-labeled metastable hairpin oligonucleotides self-assemble into long fluorescent polymers starting from an initiator sequence present on each probe molecule (Xia, et al. (2019), ibid.). The degree of amplification achieved through HCR can be tuned by changing the hybridization or polymerization times, and can be adjusted to achieve highly amplified signals (which may, however, increase the size of the fluorescent spots generated and/or lead to variable degrees of amplification for different copies of the same target molecule).

In some embodiments, provided herein are methods and compositions for analyzing analytes in a sample using concatemer primers and labeling agents. In various embodiments, a primer with domain on its 3′ end binds to a catalytic hairpin, and is extended with a new domain by a strand displacing polymerase. For example, a primer with domain 1 on its 3 ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer.

In various embodiments, a plurality of concatemer primers is contacted with a sample. In various embodiments, an assembly include a plurality of concatemer primers, a plurality of labeled probes, and a sample including nucleic acids. In various embodiments, each the plurality of concatemer primers each includes domain 1, 2, 3, etc. In various embodiments, each the plurality of labeled probes each include domain 1′, 2′, 3′, etc., with each corresponding domain 1′, 2′, 3′ being complementary to domain 1, 2, 3, etc., respectively. In various embodiments, the assembly includes the plurality of concatemer primers, which are capable of hybridizing to target nucleic acid sequences in the sample. Described herein is a method using the aforementioned assembly, including contacting the sample including target nucleic acids with the plurality of concatemer primers, then contacting the sample and plurality of concatemer primers with the plurality of labeled probes, thereby labeling the target nucleic acid sequences with a plurality of labeled probes. See e.g., Kishi et al., SABER enables amplified and multiplexes imaging of RNA and DNA in cells and tissues. Nat. Methods. (2019), Saka et al., Immuno-SABER enables highly multiplexed and amplified protein imaging in tissues. Nat. Biotechnol. (2019), and PCT Pat. Pub. No. WO 2019/147945, U.S. Pat. Pub. No. 2018/2,622,731 and U.S. Pat. Pub. No. 2018/2,622,738, each of which is fully incorporated by reference herein.

In some aspects, the provided methods comprise imaging a detectably labeled probe bound directly or indirectly to the primary probe or probe set or product thereof and detecting the detectable label. In some embodiments, the detectably labeled probe comprises a detectable label that can be measured and quantitated. The label or detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acridinium esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenicol acetyl transferase, and urease.

Fluorescence detection in tissue samples can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichloro-rhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some embodiments, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the assay comprises in situ sequencing. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932, all of which are herein incorporated by reference in their entireties. Exemplary techniques for in situ sequencing or in in situ sequence detection comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121, the content is herein incorporated by reference in its entirety).

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes can be fluorophore sequences assigned to the corresponding barcode sequences or complements thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In any of the embodiments herein, the detecting step can comprise contacting the biological sample with one or more detectably labeled probes that directly or indirectly hybridize to the barcode sequences or complements thereof (e.g., in amplification products generated using the probes or probe sets), and dehybridizing the one or more detectably labeled probes. In any of the embodiments herein, the contacting and dehybridizing steps can be repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In any of the embodiments herein, the detecting step can comprise contacting the biological sample with one or more first detectably labeled probes that directly hybridize to the plurality of probes or probe sets. In some instances, the detecting step can comprise contacting the biological sample with one or more first detectably labeled probes that indirectly hybridize to the plurality of probes or probe sets. In any of the embodiments herein, the detecting step can comprise contacting the biological sample with one or more first detectably labeled probes that directly or indirectly hybridize to the plurality of probes or probe sets.

In any of the embodiments herein, the detecting step can comprise contacting the biological sample with one or more intermediate probes that directly or indirectly hybridize to the barcode sequences or complements thereof (e.g., of the plurality of probes or probe sets or rolling circle amplification product generated using the plurality of probes or probe sets), wherein the one or more intermediate probes are detectable using one or more detectably labeled probes. In any of the embodiments herein, the detecting step can further comprise dehybridizing the one or more intermediate probes and/or the one or more detectably labeled probes from the barcode sequences or complements thereof (e.g., of the plurality of probes or probe sets or rolling circle amplification product generated using the plurality of probes or probe sets). In any of the embodiments herein, the contacting and dehybridizing steps can be repeated with the one or more intermediate probes, the one or more detectably labeled probes, one or more other intermediate probes, and/or one or more other detectably labeled probes. In some cases, the repeated contacting, detection and dehybridizing steps allows detection of barcode sequences or complements thereof and identification of the corresponding sequences of signal codes (e.g., fluorophore sequences assigned to the corresponding barcode sequences or complements thereof).

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some embodiments, the analysis and/or sequence determination involves washing to remove unbound polynucleotides, thereafter revealing a fluorescent product for imaging.

### IV. Samples, Analytes, and Target Sequences

### A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient-derived organoid (PDO) or patient-derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, needle aspirate, or fine needle aspirate. In some embodiments, the tissue sample can be from the skin, femur, pancreas, brain (e.g., cerebellum, cerebral cortex), breast, heart, kidney, large intestine, lungs, lymph node, ovaries, spleen, spinal cord, quadriceps, small intestine, stomach, testes, thyroid, tongue, or olfactory bulb. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as post-fixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ii) Embedding

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some aspects, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

(iii) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### B. Analytes

The methods provided herein allow for detection of a target nucleic acid (or a plurality of target nucleic acids) in a biological sample, which can be used to detect one or a plurality of analytes (e.g., nucleic acid or protein analytes) in the biological sample. The target nucleic acid can be a nucleic acid analyte such as an endogenous nucleic acid analyte (e.g., an mRNA), or the target nucleic acid can be associated with a nucleic acid or non-nucleic acid analyte in the biological sample. For example, the target nucleic acid can be a labeling agent as described in Section IV.B.(ii). A biological sample may comprise one or a plurality of analytes of interest. Methods for performing multiplexed assays to analyze two or more different analytes in a single biological sample are provided. The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected using the detection probes herein. In some embodiments, the methods provided herein allow for detection of any suitable target analyte using a detection probe or probe set that binds directly or indirectly to the target analyte. For example, the target analyte can be a target nucleic acid or a protein in the biological sample.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, snRNA, viral RNA, rRNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### V. Compositions and Kits

In some aspects, provided herein is a kit for analyzing a biological sample, the kit comprising: (a) a detection probe or probe set, wherein the detection probe or probe set comprises a target recognition sequence complementary to a target nucleic acid in the biological sample; (b) a ribosomal RNA (rRNA)-targeting probe or probe set, wherein the rRNA-targeting probe or probe comprises a targeting sequence complementary to a ribosomal RNA (rRNA) target sequence in the biological sample, and wherein the rRNA-targeting probe is a circular probe or a circularizable probe or probe set; and (c) a non-circularizable probe or probe set, wherein the non-circularizable probe or probe set comprises a hybridization region complementary to the rRNA target sequence or to a portion of the rRNA target sequence. In some embodiments, the detection probe or probe set is an mRNA-targeting probe or probe set that comprises a target recognition sequence complementary to a target mRNA of interest. In some aspects, provided herein is a kit for analyzing a biological sample, the kit comprising: (a) a detection probe or probe set, wherein the detection probe or probe set is capable of binding to a target analyte in the biological sample, and wherein the detection probe or probe set comprises a detectable label; (b) a ribosomal RNA (rRNA)-targeting probe or probe set, wherein the rRNA-targeting probe or probe set comprises a targeting sequence complementary to a ribosomal RNA (rRNA) target sequence in the biological sample, and wherein the rRNA-targeting probe is a circular probe or a circularizable probe or probe set; and (c) a non-circularizable probe or probe set, wherein the non-circularizable probe or probe set comprises a hybridization region complementary to the rRNA target sequence or to a portion of the rRNA target sequence. In some embodiments, the target analyte is a nucleic acid analyte or a non-nucleic acid analyte. In some embodiments, the target analyte is a protein. In some embodiments, the detection probe or probe set comprises an antibody.

In some aspects, provided herein are compositions comprising any of the rRNA-targeting probes or probe sets described herein. Also provided herein are kits, for analyzing an analyte in a biological sample according to any of the methods described herein. In some embodiments, provided herein is a kit comprising any of the rRNA-targeting probes or probe sets described herein. In some embodiments, the kit comprises a primer for performing RCA of the rRNA-targeting probe or probe set. In some embodiments, the kit comprises any of the non-circularizable probes or probe sets disclosed herein. In some embodiments, the non-circularizable probe or probe set comprises a sequence at least 95%, at least 96%, at least 97%, at least 98%, at least 99%, or 100% to the region of the a ribosomal RNA (rRNA)-targeting probe or probe set that is complementary to the target rRNA. In some embodiments, the circularizable rRNA-targeting probe and non-circularizable rRNA-targeting probe compete for binding of the rRNA target. In some embodiments, the competition between the circularizable and non-circularizable probe for hybridization reduces the number of RCA products.

In some instances, the kit further comprises any of the detection probes or probe sets disclosed herein (e.g., in Section II. C.). The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers,

### VI. Opto-Fluidic Instruments for Analysis of Biological Samples

Also provided herein is an instrument having integrated optics and fluidics modules (an “opto-fluidic instrument” or “opto-fluidic system”) for detecting target nucleic acids (e.g., nucleic acid analytes or labeling agents associated with non-nucleic acid analytes such as proteins, etc.) in biological samples (e.g., one or more cells or a tissue sample) as described herein. In an opto-fluidic instrument, the fluidics module is configured to deliver one or more reagents (e.g., detectably labeled probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles for detection and analysis (e.g., as described in Section II.D). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more analytes (e.g., as described in Section IV.B) in their naturally occurring place (i.e., in situ) within the biological sample. For example, an opto-fluidic instrument may be an in-situ analysis system used to analyze a biological sample and detect analytes including but not limited to DNA, RNA, proteins, antibodies, and/or the like. The opto-fluidic instrument may be in an in situ analysis system used to detect a target nucleic acid in the biological sample. The target nucleic acid can be a nucleic acid analyte such as an endogenous nucleic acid analyte (e.g., an mRNA), or the target nucleic acid can be associated with a nucleic acid or non-nucleic acid analyte in the biological sample. For example, the target nucleic acid can be a labeling agent as described in Section IV.B.(ii).

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

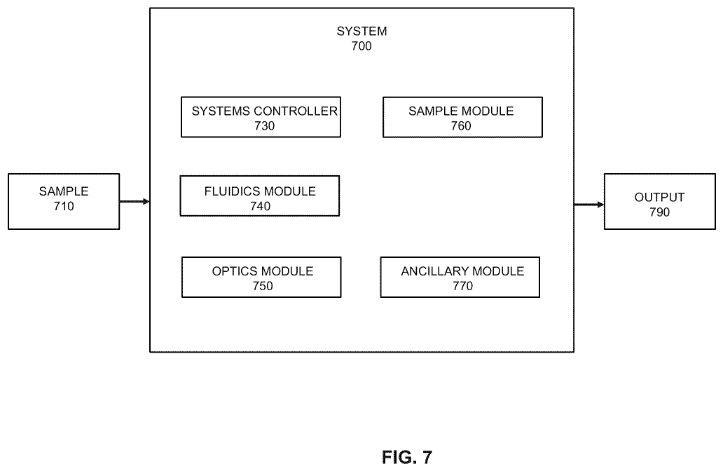

FIG. 7 shows an example workflow of analysis of a biological sample 710 (e.g., cell or tissue sample) using an opto-fluidic instrument 720, according to various embodiments. In various embodiments, the sample 710 can be a biological sample (e.g., a tissue) that includes molecules such as DNA, RNA, proteins, antibodies, etc. For example, the sample 710 can be a sectioned tissue that is treated to access the RNA thereof for labeling with probes described herein (e.g., in Section II). Ligation of the probes may generate a circular probe which can be enzymatically amplified and bound with detectably labeled probes, which can create bright signal that is convenient to image and has a high signal-to-noise ratio.

In various embodiments, the sample 710 may be placed in the opto-fluidic instrument 720 for analysis and detection of the molecules in the sample 710. In various embodiments, the opto-fluidic instrument 720 can be a system configured to facilitate the experimental conditions conducive for the detection of the target molecules. For example, the opto-fluidic instrument 720 can include a fluidics module 740, an optics module 750, a sample module 760, and an ancillary module 770, and these modules may be operated by a system controller 730 to create the experimental conditions for the probing of the molecules in the sample 710 by selected probes (e.g., circularizable DNA probes), as well as to facilitate the imaging of the probed sample (e.g., by an imaging system of the optics module 750). In various embodiments, the various modules of the opto-fluidic instrument 720 may be separate components in communication with each other, or at least some of them may be integrated together.

In various embodiments, the sample module 760 may be configured to receive the sample 710 into the opto-fluidic instrument 720. For instance, the sample module 760 may include a sample interface module (SIM) that is configured to receive a sample device (e.g., cassette) onto which the sample 710 can be deposited. That is, the sample 710 may be placed in the opto-fluidic instrument 720 by depositing the sample 710 (e.g., the sectioned tissue) on a sample device that is then inserted into the SIM of the sample module 760. In some instances, the sample module 760 may also include an X-Y stage onto which the SIM is mounted. The X-Y stage may be configured to move the SIM mounted thereon (e.g., and as such the sample device containing the sample 710 inserted therein) in perpendicular directions along the two-dimensional (2D) plane of the opto-fluidic instrument 720.

The experimental conditions that are conducive for the detection of the molecules in the sample 710 may depend on the target molecule detection technique that is employed by the opto-fluidic instrument 620. For example, in various embodiments, the opto-fluidic instrument 720 can be a system that is configured to detect molecules in the sample 710 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 710 using reagents such as washing/stripping reagents, hybridizing reagents, etc., and such reagents may be provided by the fluidics module 740.

In various embodiments, the fluidics module 740 may include one or more components that may be used for storing the reagents, as well as for transporting said reagents to and from the sample device containing the sample 710. For example, the fluidics module 740 may include reservoirs configured to store the reagents, as well as a waste container configured for collecting the reagents (e.g., and other waste) after use by the opto-fluidic instrument 720 to analyze and detect the molecules of the sample 710. Further, the fluidics module 740 may also include pumps, tubes, pipettes, etc., that are configured to facilitate the transport of the reagent to the sample device (e.g., and as such the sample 710). For instance, the fluidics module 740 may include pumps (“reagent pumps”) that are configured to pump washing/stripping reagents to the sample device for use in washing/stripping the sample 710 (e.g., as well as other washing functions such as washing an objective lens of the imaging system of the optics module 750).

In various embodiments, the ancillary module 770 can be a cooling system of the opto-fluidic instrument 720, and the cooling system may include a network of coolant-carrying tubes that are configured to transport coolants to various modules of the opto-fluidic instrument 720 for regulating the temperatures thereof. In such cases, the fluidics module 740 may include coolant reservoirs for storing the coolants and pumps (e.g., “coolant pumps”) for generating a pressure differential, thereby forcing the coolants to flow from the reservoirs to the various modules of the opto-fluidic instrument 720 via the coolant-carrying tubes. In some instances, the fluidics module 740 may include returning coolant reservoirs that may be configured to receive and store returning coolants, i.e., heated coolants flowing back into the returning coolant reservoirs after absorbing heat discharged by the various modules of the opto-fluidic instrument 720. In such cases, the fluidics module 740 may also include cooling fans that are configured to force air (e.g., cool and/or ambient air) into the returning coolant reservoirs to cool the heated coolants stored therein. In some instance, the fluidics module 740 may also include cooling fans that are configured to force air directly into a component of the opto-fluidic instrument 720 so as to cool said component. For example, the fluidics module 740 may include cooling fans that are configured to direct cool or ambient air into the system controller 730 to cool the same.

As discussed above, the opto-fluidic instrument 720 may include an optics module 750 which include the various optical components of the opto-fluidic instrument 720, such as but not limited to a camera, an illumination module (e.g., LEDs), an objective lens, and/or the like. The optics module 750 may include a fluorescence imaging system that is configured to image the fluorescence emitted by the probes (e.g., oligonucleotides) in the sample 710 after the probes are excited by light from the illumination module of the optics module 750.

In some instances, the optics module 750 may also include an optical frame onto which the camera, the illumination module, and/or the X-Y stage of the sample module 760 may be mounted.

In various embodiments, the system controller 730 may be configured to control the operations of the opto-fluidic instrument 720 (e.g., and the operations of one or more modules thereof). In some instances, the system controller 730 may take various forms, including a processor, a single computer (or computer system), or multiple computers in communication with each other. In various embodiments, the system controller 730 may be communicatively coupled with data storage, set of input devices, display system, or a combination thereof. In some cases, some or all of these components may be considered to be part of or otherwise integrated with the system controller 730, may be separate components in communication with each other, or may be integrated together. In other examples, the system controller 730 can be, or may be in communication with, a cloud computing platform.

In various embodiments, the opto-fluidic instrument 720 may analyze the sample 710 and may generate the output 790 that includes indications of the presence of the target molecules in the sample 710. For instance, with respect to the example embodiment discussed above where the opto-fluidic instrument 720 employs a hybridization technique for detecting molecules, the opto-fluidic instrument 720 may cause the sample 710 to undergo successive rounds of detectably labeled probe hybridization (e.g., using two or more sets of fluorescent probes, where each set of fluorescent probes is excited by a different color channel) and be imaged to detect target molecules in the probed sample 710. In such cases, the output 790 may include optical signatures (e.g., a codeword) specific to each gene, which allow the identification of the target molecules.

### VII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se. In some instances, the term “about” can refer to a value within 20% of an indicated value. In some instances, the term “about” can refer to a value within 10% of an indicated value.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: RCA of 18S rRNA-Targeting Probes Results in Expanded Cellular Morphology

This example provides results demonstrating expansion of cellular morphology after RCA of circularizable padlock probes targeting 18S rRNA in a biological sample. Mixtures of 18S rRNA-targeting probes comprising circularizable probes and competing non-circularizable probes at different ratios were used to control the number of RCA products (RCPs) produced using the circularizable probes. The circularizable probes comprised a 5′ arm and a 3′ arm complementary to a 18S rRNA target sequence, such that upon hybridization of the circularizable probe to the 18S rRNA target sequence the 5′ end and 3′ end were juxtaposed for ligation. The circularizable probes had a ligatable 5′ end comprising a 5′ phosphate. For this experiment, the non-circularizable probes had the same sequence as the circularizable probes, but lacked a ligatable 5′ phosphate.

Probe mixtures were tested at various ratios of circularizable 18S rRNA-targeting probes to total (circularizable and non-circularizable) 18S rRNA targeting probes (e.g., 1:2, 1:4, 1:10, 1:25, 1:50) in situ within mouse brain samples for quantification of cellular expansion. At a ratio of 1:10 circularizable 18S rRNA-targeting probes to total (circularizable and non-circularizable) 18S rRNA-targeting probes, cellular morphology was expanded while the nucleus remained intact. Unexpectedly, performing RCA of circularized probes targeting 18S rRNA achieved an expansion-microscopy-like effect and improved resolution of optical signals in situ.

Mouse brain tissue sections (e.g., tissue slices) were prepared by fixation followed by permeabilization and washed. A mixture comprising circularizable probes and non-circularizable probes targeting 18S rRNA were contacted with the tissue sections and allowed to hybridize in hybridization buffer overnight at 37° C. Hybridized circularizable probes were then ligated to circularize the circularizable probes. For RCA, the section was contacted with an RCA mixture, containing Phi29 polymerase buffer, dNTPs, RCA primer and Phi29 polymerase and incubated. Subsequently, the section was washed in PBS-T twice. Primary HH3 antibody was added. Tissue sections were fixed. Sections were incubated with blocking buffer, then intermediate probes (each with a region for hybridizing to a sequence of the RCP and an overhang region for hybridizing to fluorescently-labeled probes) were hybridized in hybridization buffer at 34° C. for 30 minutes, followed by application of a secondary HH3 antibody and imaging. Then-fluorescently-labeled probes were hybridized at 30° C. for 30 minutes for detecting the RCPs associated with 18S rRNA. Sections were imaged using a fluorescent microscope with a 20× objective, and the area of the dentate gyrus was quantified.

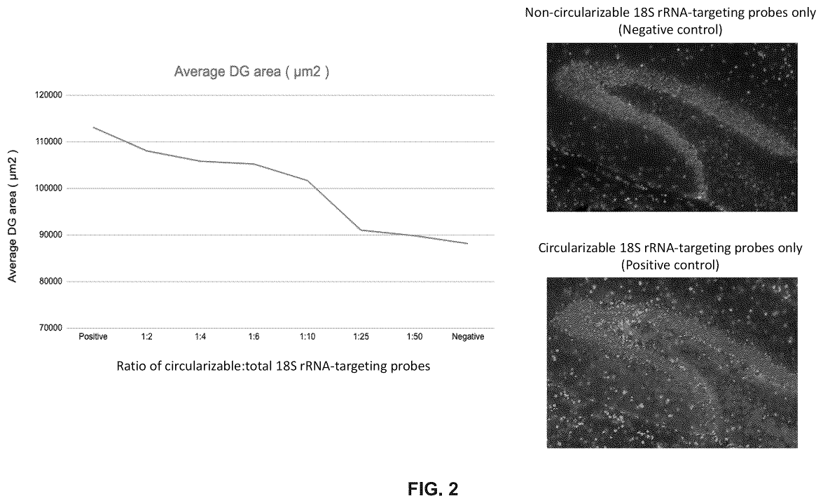

Performing RCA of circularized probes targeting 18S rRNA resulted in expansion of the tissue and of cellular morphology. Notably, the expansion effect could be controlled by varying the ratios of circularizable 18S rRNA-targeting probes and non-circularizable 18S rRNA-targeting probes in the probe mixture. FIG. 2 provides quantification of average dentate gyrus area at various ratios of circularizable to total (circularizable and non-circularizable) 18S rRNA probe. These results show a concentration-dependent (e.g. ratio-dependent) correlation between the concentration of circularizable 18S rRNA probe (e.g. in relation to total 18S rRNA probe) and the average area of the dentate gyrus. The area of the dentate gyrus was expanded up to 28% in the positive control (5 nM circularizable 18S rRNA-targeting probe only, without non-circularizable 18S rRNA-targeting probe) (Table 1). The right panel of FIG. 2 provides fluorescent images of the negative control dentate gyrus (100% non-circularizable 18S rRNA-targeting probe, and treated with a phosphatase to ensure there were no phosphate group remaining in 5′ end) and positive control (100% circularizable 18S rRNA-targeting probe), showing the expanded morphology of cells that underwent RCA with circularizable 18S rRNA probes.

TABLE 1
Percent difference in average dentate gyrus area
compared to negative control (non-circularizable
18S rRNA-targeting probe) at various probe ratios

| Ratio of Circularizable 18S rRNA- | |
|---|---|
| targeting probe to total | Difference in dentate |
| (circularizable and non-circularizable) | gyrus average area compared |
| 18S rRNA-targeting probe | to negative control (%) |
| Circularizable probe only (1:1) | 28.35 |
| 1:2 | 22.61 |
| 1:4 | 20.07 |
| 1:6 | 19.40 |
| 1:10 | 15.38 |
| 1:25 | 3.29 |
| 1:50 | 1.91 |

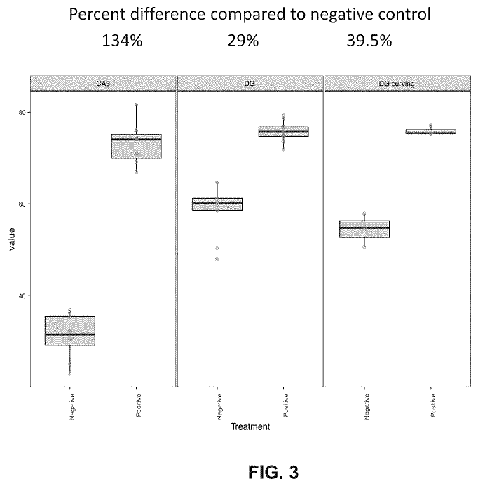

The expanded morphology of cells that underwent RCA with 18S rRNA probes were further characterized as shown in FIG. 3, where the thickness of the dentate gyrus, the CA3 region, and the curvature of the dentate gyrus were measured. Compared to the negative control, the thickness of the CA3 region increased by 134%, the thickness of the dentate gyrus increased by 29%, and the thickness of the curvature of the dentate gyrus increased by 39.5%. Together, these results suggest that the expansion occurred in all directions of the tissue (e.g. isometrically).

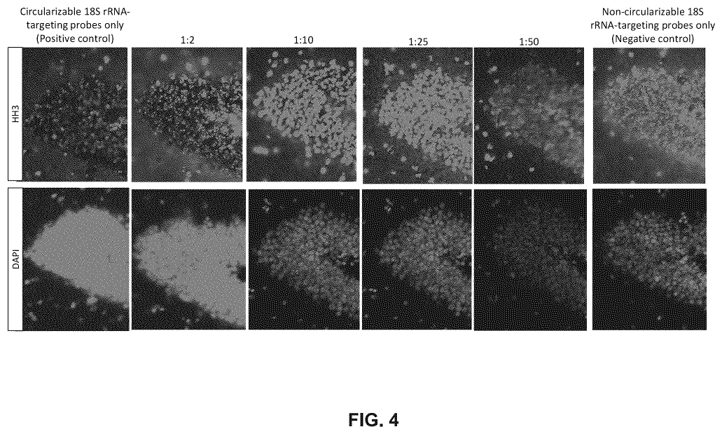

To investigate the effect of the RCA-dependent expansion on cellular structures, costaining with DAPI and HH3 was performed in tissue sections that underwent RCA with various ratios of circularizable:total 18S rRNA-targeting probes. The results are shown in the fluorescent images in FIG. 4. It was observed that while HH3 always costained with the DAPI nucleus staining in the negative control with only non-circularizable 18S rRNA probes, costaining was greatly reduced in the positive control with only circularizable 18S rRNA probes. This suggests that the nucleus was disrupted in the positive control. However, at ratios of circularizable:total 18S rRNA-targeting probes of 1:10 or lower (i.e., 1:25, 1:50) HH3-DAPI costaining was restored, and analysis confirmed that cell segmentation was performed accurately at 1:10 ratio of circularizable:total 18S rRNA-targeting probes. These results demonstrate that the ratio of competing circularizable and non-circularizable 18S rRNA-targeting probes can be varied to control the degree of cell expansion, thus achieving expansion of cells (FIG. 2) while retaining intact nuclei (FIG. 4) and/or other cellular structures.

### Example 2: RCA of Circularized Non-Detected 18S rRNA-Targeting Probes Increases Sensitivity for Detection of Target RNAs

This example provides results demonstrating increasing signal sensitivity by using a mixture of circularizable and non-circularizable padlock probes targeting 18S rRNA to generate RCPs in a biological sample. In some aspects, the RCPs generated from circularized probes targeting 18s rRNA produced a physical expansion effect in the biological sample.

Mouse brain tissue sections were prepared as in Example 1 above. 18S rRNA-targeting probe mixtures comprising competing circularizable and non-circularizable probes were also contacted with the tissue sections as in Example 1 above. In this Example, the tissue sections were also contacted with a panel of circularizable detection probes, comprising detection probes targeting a plurality of different target mRNAs (a panel of 248 targets including mouse brain genes), and containing barcode sequences corresponding to their target mRNAs. Ligation and RCA of the circularizable probes were performed as described in Example 1 above. Following RCA, the complementary barcode sequences of RCPs produced from the circularized detection probes were decoded using sequential hybridization and detection of a series of panels of intermediate and fluorescently-labeled probes. The RCPs produced from circularizable 18S rRNA-targeting probes were not detected.

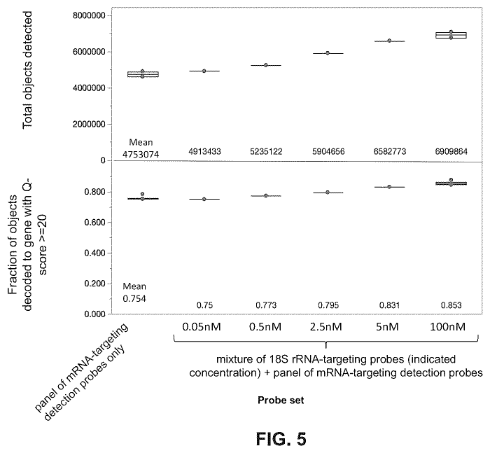

FIG. 5 shows the total number of objects (top) and fraction of objects that were detected in tissue sections that underwent RCA with the mixture of 18S rRNA-targeting probes indicated and the panel of mRNA-targeting detection probes. Above a 1:100 ratio of circularizable (0.05 nM) to total (circularizable and non-circularizable) 18S rRNA-targeting probes, the number of targets of the detection probe panel that were detected increased with increasing concentrations of the circularizable 18S rRNA probe. This suggests that generating RCPs of the circularized 18S rRNA probe in the biological sample improved sensitivity of the detection of the target nucleic acid. At a 1:10 ratio of circularizable to total (circularizable and non-circularizable) 18S rRNA-targeting probes, specificity is maintained and a 33-37% increase in sensitivity was observed with no disruption to nuclear morphology.

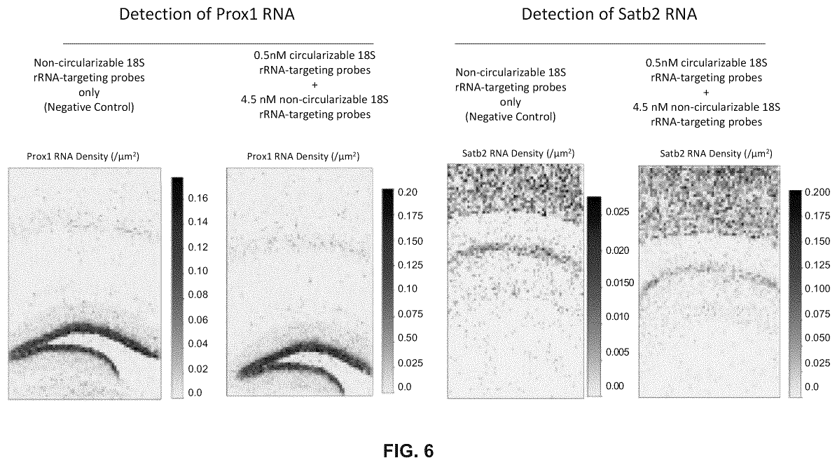

FIG. 6 provides expression heatmaps showing distribution and density of decoded barcodes corresponding to Prox1 and Satb2 RNA, two representative mRNA targets from the detection probe panel with well-characterized expression patterns. Prox1 and Satb2 detected expression heatmaps are shown after RCA of the detection probe panel together with either non-circularizable 18S rRNA-targeting probes (negative control) or a 1:10 ratio of circularizable:total (circularizable and non-circularizable) 18S rRNA-targeting probe mixture. The detected expression patterns of Prox1 and Satb2 were consistent in the samples with circularizable 18S rRNA-targeting probes and in the negative control. These results demonstrate that the increased sensitivity in detection of RNA targets using RCA of circularizable 18S rRNA-targeting probes does not impact specificity. By tuning the optimal ratio of probes used, the cell area was increased, showing an expansion-microscopy-like effect while maintaining the nucleus morphology (e.g., biologically intact and not deformed morphology). In some cases, it was observed that nucleus and cellular morphology was expanded especially at the denser cell areas but the physiology of the cellular features were preserved and restored with increased overall dimensional thickness allowing benefits of an expansion-microscopy-like effect.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method, comprising:
(a) contacting a biological sample with a detection probe or probe set, wherein the detection probe or probe set binds to a target analyte in the biological sample;
(b) contacting the biological sample with a ribosomal RNA (rRNA)-targeting probe or probe set, wherein the rRNA-targeting probe or probe set hybridizes to a ribosomal RNA (rRNA) target sequence in the biological sample, and wherein the rRNA-targeting probe is a circular probe or the rRNA-targeting probe or probe set is a circularizable probe or probe set and the method comprises circularizing the rRNA-targeting probe or probe set;
(c) performing rolling circle amplification (RCA) of the rRNA-targeting probe or probe set to generate a non-detection rolling circle amplification product (RCP) in the biological sample; and
(d) imaging the biological sample to detect an optical signal associated with the detection probe or probe set or a product thereof, without detecting an optical signal associated with the non-detection RCP.

2. The method of claim 1, wherein:
the target analyte is a target nucleic acid;
the detection probe or probe set hybridizes to the target nucleic acid; and
the detection probe or probe set is a circular probe, or the detection probe or probe set is a circularizable probe or probe set and the method comprises circularizing the detection probe or probe set.

3. The method of claim 2, wherein the method comprises performing RCA of the circular or circularized detection probe or probe set to generate a detection RCP, and the optical signal detected in (d) is associated with the detection RCP.

4-9. (canceled)

10. The method of claim 3, wherein a sequence of the detection RCP is analyzed at a location in the biological sample or a matrix embedding the biological sample.

11-14. (canceled)

15. The method of claim 1, wherein the biological sample is a eukaryotic sample, and wherein the rRNA is selected from the group consisting of 5S rRNA, 18S rRNA, 28S rRNA, and 5.8S rRNA.

16. The method of claim 15, wherein the rRNA is 18S rRNA.

17-19. (canceled)

20. The method of claim 1, wherein the method comprises contacting the biological sample with a non-circularizable probe or probe set, wherein the non-circularizable probe or probe set comprises a hybridization region complementary to the rRNA target sequence or to a portion of the rRNA target sequence.

21. The method of claim 20, wherein a 5′ end of the non-circularizable probe or probe set lacks a 5′ phosphate.

22-27. (canceled)

28. The method of claim 20, wherein the rRNA-targeting probe or probe set and the non-circularizable probe or probe set are contacted with the biological sample simultaneously.

29. The method of claim 20, wherein the ratio of the concentration of the rRNA-targeting probe or probe set to the total concentration of the rRNA-targeting probe set and the non-circularizable probe or probe set is about 1:2 or lower.

30-42. (canceled)

43. The method of claim 1, wherein the method does not comprise analyzing a sequence of the non-detection RCP at a location in the biological sample or a matrix embedding the biological sample.

44. (canceled)

45. The method of claim 1 wherein the target analyte is a cellular nucleic acid analyte or a product thereof.

46. The method of claim 1, wherein the target analyte is a protein, or wherein the target analyte is an oligonucleotide reporter in a labeling agent that binds to a protein.

47-62. (canceled)

63. The method of claim 1,
wherein the volume of a cell in the biological sample after the RCA of the rRNA-targeting probe or probe set (V2) is greater than the volume of the cell before the RCA of the rRNA-targeting probe or probe set (V1).

64-72. (canceled)

73. The method of claim 1, wherein the biological sample is a tissue section, and wherein an area of the tissue section after the RCA of the rRNA-targeting probe or probe set (A2) is greater than the area of the tissue section before the RCA of the rRNA-targeting probe or probe set (A1).

74. The method of claim 73, wherein A2 is at least 105% of A1.

75-77. (canceled)

78. The method of claim 1, wherein the method results in an increase in sensitivity for detection of the target analyte in comparison to a control biological sample that is not contacted with the circular or circularizable rRNA-targeting probe or probe set.

79-127. (canceled)

128. The method of any of claim 1, wherein (a) is performed before, after, or simultaneously with (b).

129-131. (canceled)

132. A method of detecting a target analyte in a cell, comprising:
(a) using a circular or circularizable ribosomal RNA (rRNA)-targeting probe or probe set to generate a plurality of non-detection rolling circle amplification products (RCPs) in the cell, thereby increasing the volume of the cell; and
(b) detecting an optical signal associated with the target analyte.

133-141. (canceled)

142. A kit for analyzing a biological sample, the kit comprising:
(a) a detection probe or probe set, wherein the detection probe or probe set is capable of binding to a target analyte in the biological sample;
(b) a ribosomal RNA (rRNA)-targeting probe or probe set, wherein the rRNA-targeting probe or probe set comprises a targeting sequence complementary to a ribosomal RNA (rRNA) target sequence in the biological sample, and wherein the rRNA-targeting probe is a circular probe or a circularizable probe or probe set; and
(c) a non-circularizable probe or probe set, wherein the non-circularizable probe or probe set comprises a hybridization region complementary to the rRNA target sequence or to a portion of the rRNA target sequence.

143-145. (canceled)
